(19) United States
(12) Patent Application Publication
(10) Pub. No.: US 2021/0386915 A1
(43) Pub. Date: Dec. 16, 2021

(54) COMPOSITIONS AND METHODS FOR AIRWAY TISSUE REGENERATION

(71) Applicant: The Board of Trustees of the Leland Stanford Junior University, Stanford, CA (US)

(72) Inventors: Dawn T. BRAVO, Stanford, CA (US); Sriram VAIDYANATHAN, Stanford, CA (US); Matthew H. PORTEUS, Stanford, CA (US); Calvin J. KUO, Stanford, CA (US); Jayakar NAYAK, Stanford, CA (US); Ameen SALAHUDEEN, Stanford, CA (US); Tushar J. DESAI, Stanford, CA (US)

(73) Assignee: The Board of Trustees of the Leland Stanford Junior University, Stanford, CA (US)

(21) Appl. No.: 17/353,049

(22) Filed: Jun. 21, 2021

Related U.S. Application Data
(63) Continuation of application No. PCT/US2019/067486, filed on Dec. 19, 2019.
(60) Provisional application No. 62/784,125, filed on Dec. 21, 2018.

(51) Int. Cl. A61L 27/38; C12N 9/22; C12N 15/11; C12N 15/90; A61L 27/36

(52) U.S. Cl. CPC A61L 27/3834; C12N 9/22; C12N 2310/20; C12N 15/907; A61L 27/3633; C12N 15/111

(57) ABSTRACT

The present disclosure provides compositions and methods for regenerating airway stem cells, as well as methods for treating an airway disease (e.g., cystic fibrosis (CF)) in a subject using the regenerated airway stem cells.

### CROSS-REFERENCE TO RELATED APPLICATIONS

This application is a continuation of International Application No. PCT/US2019/067486 filed Dec. 19, 2019, which claims priority to U.S. Provisional Application No. 62/784,125, filed Dec. 21, 2018, the disclosure of which is hereby incorporated by reference in its entirety for all purposes.

### BACKGROUND

Cystic fibrosis (CF) is a monogenic disorder caused by mutations in the Cystic Fibrosis Transmembrane Conductance Regulator (CFTR) Cl⁻ channel, resulting in multi-organ dysfunction and ultimately mortality from respiratory sequelae. Although CF is a systemic disease that affects multiple organ systems, CF lung disease is the major cause of morbidity and mortality in CF patients. Over the past decade, small molecule CFTR correctors and potentiators have been developed, and represent a significant advancement in CF therapeutics.^{1,2} Although these small molecules improve lung function and reduce pulmonary exacerbations and respiratory decline in patients, they are expensive, show variable therapeutic responses, have adverse effects (e.g., hepatotoxicity), and must be administered daily.³ As a result, there is continued interest in developing genome editing strategies to correct CFTR mutations and achieve durable restoration of native CFTR function.

Genome editing using zinc-finger nucleases or CRISPR/Cas9 has been attempted in intestinal cells and induced pluripotent stem cells (iPSCs), respectively.⁴⁻⁶ These studies focused on the ΔF508 mutation that affects >70% of CFTR patients and reported the use of selectable markers to enrich for edited cells. The efficiencies reported in these studies, 0.02%⁴ before selection to 6% after selection⁵, although low, are useful to understand the pathophysiology of different mutations and may enable drug screening. In addition to poor correction efficiencies both in vitro and in vivo, the process to differentiate iPSCs into the appropriate clinically relevant airway phenotype is still uncertain. In contrast, a rapid, highly efficient selection-free strategy in endogenous airway stem cells could enable the development of cell therapies to treat CF and other airway diseases.

### SUMMARY

In one aspect, the disclosure features a composition for airway tissue regeneration, comprising an airway stem cell and a bioscaffold (e.g., a decellularized extracellular matrix (ECM) membrane), wherein the airway stem cell expresses cytokeratin 5 (Krt5) and is embedded in the bioscaffold (e.g., the decellularized ECM membrane). In some embodiments, the bioscaffold comprises a decellularized ECM membrane and/or collagen Type-I.

In some embodiments, the airway stem cell expresses a wild-type Cystic Fibrosis Transmembrane Conductance Regulator (CFTR) protein. In some embodiments, the airway stem cell is an upper airway stem cell, such as an upper airway basal stem cell (e.g., a sinus basal stem cell). In some embodiments, the airway stem cell is a lower airway stem cell, such as a bronchial stem cell (e.g., a human bronchial epithelial cell (HBEC)). As used herein, the terms “basal cell” and “basal stem cell” are used interchangeably.

In some embodiments, the airway stem cell is a gene edited airway stem cell. A gene edited airway stem cell may be gene edited to correct an amino acid mutation in a protein (e.g., a CFTR protein). In particular embodiments, the gene edited airway stem cell is gene edited to correct an amino acid mutation at position 508 of a mutated CFTR protein. The gene edited airway stem cell may be gene edited using a CRISPR (Clustered Regularly Interspaced Short Palindromic Repeats)/Cas (CRISPR-associated protein) nuclease system.

In some embodiments, the composition further comprises airway ciliated cells (e.g., airway ciliated cells expressing acetylated alpha tubulin) and/or airway mucus producing cells (e.g., airway mucus producing cells expressing MUC5AC).

In some embodiments, the bioscaffold is a decellularized ECM membrane. The decellularized ECM membrane may be derived from a tissue source selected from the group consisting of interstine tissue, pancreas tissue, liver tissue, lung tissue, trachea tissue, esophagus tissue, kidney tissue, bladder tissue, skin tissue, heart tissue, brain tissue, placenta tissue, and umbilical cord tissue. In some embodiments, the decellularized ECM membrane is derived from a mammalian tissue source. In particular embodiments, the decellularized ECM membrane is a porcine small intestinal submucosal (pSIS) membrane.

In another aspect, the disclosure features a method for airway tissue regeneration, comprising: (a) inducing a stable gene modification of a target nucleic acid encoding a mutated protein in an airway stem cell via homologous recombination by introducing into the airway stem cell: (1) a single guide RNA (sgRNA) comprising a first nucleotide sequence that is complementary to the target nucleic acid, and a second nucleotide sequence that interacts with a CRISPR-associated protein (Cas) polypeptide; (2) a Cas polypeptide, an mRNA encoding a Cas polypeptide, and/or a recombinant expression vector comprising a nucleotide sequence encoding a Cas polypeptide, wherein the sgRNA guides the Cas polypeptide to the target nucleic acid; and (3) a homologous donor adeno-associated viral (AAV) vector comprising a recombinant donor template comprising two nucleotide sequences comprising two non-overlapping, homologous portions of the target nucleic acid, wherein the nucleotide sequences are located at the 5′ and 3′ ends of a nucleotide sequence corresponding to the target nucleic acid to undergo homologous recombination; (b) embedding the airway stem cell in a bioscaffold (e.g., a decellularized extracellular matrix (ECM) membrane); and (c) culturing the airway stem cell embedded in the bioscaffold (e.g., the decellularized ECM membrane), wherein the airway stem cell expresses Krt5. In some embodiments, the bioscaffold comprises a decellularized ECM membrane and/or collagen Type-I.

In some embodiments of the method, the mutated protein is a mutated CFTR protein and the target nucleic acid is modified to encode a corresponding wild-type CFTR protein of the mutated CFTR protein in step (a). In some embodiments, the mutated CFTR protein does not have a phenylalanine (F) at position 508. In some embodiments, the airway stem cell embedded in the bioscaffold (e.g., the decellularized ECM membrane) differentiates into airway ciliated cells (e.g., airway ciliated cells expressing acetylated alpha tubulin) and/or airway mucus producing cells (e.g., airway mucus producing cells expressing MUC5AC).

In some embodiments of the method, the homologous donor AAV vector is selected from a wild-type AAV serotype 1 (AAV1), wild-type AAV serotype 2 (AAV2), wild-type AAV serotype 3 (AAV3), wild-type AAV serotype 4 (AAV4), wild-type AAV serotype 5 (AAV5), wild-type AAV serotype 6 (AAV6), wild-type AAV serotype 7 (AAV7), wild-type AAV serotype 8 (AAV8), wild-type AAV serotype 9 (AAV9), wild-type AAV serotype 10 (AAV10), wild-type AAV serotype 11 (AAV11), wild-type AAV serotype 12 (AAV12), a variant thereof, and any shuffled chimera thereof. In some embodiments, the homologous donor AAV vector is a wild-type AAV6 or an AAV6 variant having at least 95% sequence identity to wild-type AAV6.

In some embodiments of the method, the airway stem cell comprises a population of airway stem cells. In some embodiments, the stable gene modification of the target nucleic acid is induced in greater than about 70% (e.g., 72%, 74%, 76%, 78%, 80%, 82%, 84%, 86%, 88%, 90%, 92%, 94%, 96%, 98%, or 100%) of the population of airway stem cells.

In some embodiments of the method, the Cas polypeptide is a Cas9 polypeptide, a variant thereof, or a fragment thereof. In some embodiments, the sgRNA comprises at least one modified nucleotide. In some embodiments of the method, the sgRNA is used to correct a ΔF508 mutation in the mutated CFTR protein. In particular embodiments of the method, the sgRNA comprises a sequence having at least 80% sequence identity (e.g., 82%, 85%, 90%, 91%, 92%, 93%, 94% 95%, 96%, 97%, 98%, or 99% sequence identity) to a sequence of UCUGUAUCUAUAUUCAUCAU (SEQ ID NO: 1). In some embodiments, the sgRNA comprises a sequence having at least one nucleotide substitution (e.g., two, three, or four amino acid mutations) relative to the sequence of UCUGUAUCUAUAUUCAUCAU (SEQ ID NO: 1).

In some embodiments of the method, the sgRNA and the Cas polypeptide are incubated together to form a ribonucleoprotein (RNP) complex prior to introducing into the airway stem cell. The RNP complex and the homologous donor AAV vector may be concomitantly introduced into the airway stem cell. In some embodiments, the RNP complex and the homologous donor AAV vector may be sequentially introduced into the airway stem cell. The RNP complex may be introduced into the airway stem cell before the homologous donor AAV vector. The RNP complex may be introduced into the airway stem cell after the homologous donor AAV vector.

In some embodiments of the method, the homologous donor AAV vector carries a sequence having at least 80% sequence identity (e.g., 82%, 85%, 90%, 91%, 92%, 93%, 94% 95%, 96%, 97%, 98%, or 99% sequence identity) to a sequence of SEQ ID NO: 10.

In some embodiments of the method, the sgRNA and the Cas polypeptide are introduced into the airway stem cell via electroporation. In some embodiments, the homologous donor AAV vector is introduced into the airway stem cell via transduction.

In another aspect, the disclosure features, ex vivo regenerated airway stem cells produced by the method described above.

In another aspect, the disclosure features a method for treating an airway disease in a subject having a mutated protein, comprising grafting a composition comprising an airway stem cell and a bioscaffold (e.g., a decellularized ECM membrane), wherein the mutated protein causes the airway disease, the airway stem cell expresses Krt5 and a corresponding wild-type protein of the mutated protein, and the airway stem cell is embedded in the bioscaffold (e.g., the decellularized ECM membrane). In some embodiments, the bioscaffold comprises a decellularized ECM membrane and/or collagen Type-I.

In some embodiments of this method, the airway disease is cystic fibrosis (CF). In some embodiments, the mutated protein is a mutated CFTR protein. In particular embodiments, the mutated CFTR protein does not have a phenylalanine (F) at position 508.

In some embodiments of this method, the method further comprises, prior to the grafting, isolating an airway stem cell from the subject having the mutated protein and gene editing the isolated airway stem cell to express a corresponding wild-type protein of the mutated protein. Further the method comprises embedding the gene edited airway stem cell expressing the corresponding wild-type protein in the bioscaffold (e.g., the decellularized ECM membrane). In some embodiments of this method, the gene edited airway stem cell is edited using CRISPR (Clustered Regularly Interspaced Short Palindromic Repeats)/Cas (CRISPR-associated protein) nuclease system.

In some embodiments of this method, the method further comprises, prior to the grafting, embedding the airway stem cell expressing Krt5 and the corresponding wild-type protein of the mutated protein in the bioscaffold (e.g., the decellularized ECM membrane).

In some embodiments of this method, the airway disease is selected from the group consisting of cystic fibrosis, chronic bronchitis, ciliary dyskinesia, bronchiectasis, chronic occlusive pulmonary disease (COPD), and diffuse panbronchiolitis. In particular embodiments, the airway disease is cystic fibrosis.

In some embodiments of this method, the airway stem cell is an upper airway stem cell, such as an upper airway basal stem cell (e.g., a sinus basal stem cell). In some embodiments, the airway stem cell is a lower airway stem cell, such as a bronchial stem cell (e.g., a human bronchial epithelial cell (HBEC)).

In some embodiments of this method, the bioscaffold is a decellularized ECM membrane. The decellularized ECM membrane may be derived from a tissue source selected from the group consisting of interstine tissue, pancreas tissue, liver tissue, lung tissue, trachea tissue, esophagus tissue, kidney tissue, bladder tissue, skin tissue, heart tissue, brain tissue, placenta tissue, and umbilical cord tissue. The decellularized ECM membrane may be derived from a mammalian tissue source. In particular embodiments, the decellularized ECM membrane is a porcine small intestinal submucosal (pSIS) membrane.

### BRIEF DESCRIPTION OF THE DRAWINGS

FIG. 1A: Representative traces obtained from epithelial sheets derived from CF bronchial basal cells of ΔF508 homozygous (ΔF/ΔF).

FIG. 1B: Correction of ΔF508 mutation in 30% alleles resulted in a restoration of CFTR function.

FIG. 2A: Percentage of Krt5⁺ cells on day 0, day 5, and fold expansion observed in 6 subjects.

FIGS. 2B and 2C: Representative FACS plots show Krt5⁺ gate on day 0 and day 5.

FIGS. 2D and 2E: Optimal proliferation was observed at cells densities between 10,000-20000 cells/cm² both at P.0 and P.1

FIG. 2F: Culturing cells in 5% 02 improved proliferation of cells from ⅔ subjects.

FIG. 3A: Sinus cells were cultured as organoids in Matrigel (image 1 and 2). Organoids were assessed by H&E stains (Image 3). Organoids were positive for Krt5 on the day of editing (day 5, image 4).

FIG. 3B: Gene editing was performed on cells cultured both as monolayers and organoids. Editing efficiencies were higher for cells cultured as organoids.

FIG. 4A: AAV6 showed the best transduction in airway basal cells. Cells were transduced within 5 minutes after electroporation.

FIG. 4B: In cells obtained from non-CF patients, MOIs of 10⁶ and 2*10⁶ M vector genomes (vg)/cell showed significantly higher editing compared to MOIs <2*10⁵ vg/cell. Different symbols represent cells from a different donor.

FIGS. 4C and 4D: HR templates with silent mutations on both sides of the DSB site resulted in higher HR than templates containing mutations on one side.

FIG. 5A: Schematic describing the Cas9/AAV mediated strategy to correct ΔF508. The underlined segment represents the sequence complementary to sgRNA used. The PAM (protospacer adjacent motif) is indicated in bold for the wild-type (WT) sequence. Silent mutations introduced in the correction template are colored in green.

FIG. 5B: The region around exon 11 was amplified using IN-OUT PCR to quantify INDELS and HR using TIDER. INDELs were observed in 38±2% alleles and HR was observed in 43±5% alleles. Controls treated with only AAV did not show any INDELs or HR.

FIGS. 5C and 5D: On day 4 after editing, the cells were stained for Krt5 and Integrin alpha 6 (ITGA6). The Krt5⁺ITGA6⁺ population is similar between control and edited cells.

FIG. 6A: Summary of % alleles exhibiting HR in CF patient samples (ΔF/ΔF—homozygous and ΔF/other—Compound heterozygous).

FIG. 6B: Western blot probing CFTR expression. Calu-3 cells were used a positive controls (lane 1). WT nasal cells (lane 2) showed a clear band corresponding to the mature CFTR. Mature CFTR expression was absent in ΔF508 homozygous (ΔF/ΔF) cells (lane 3) but a faint band corresponding to immature CFTR was present (CFTR Band B). ΔF508 homozygous (ΔF/ΔF) cells after correction showed a restored mature CFTR band (lane 4).

FIGS. 6C and 6D: Representative traces obtained from epithelial sheets by Ussing chamber analysis.

FIG. 6E: CFTRᵢₙₕ-172 sensitive short circuit currents observed in non-CF, uncorrected and corrected CF sinus samples as a function of editing (ΔF/ΔF: n=4 donors, ΔF/other: n=3 donors and non-CF: n=3 donors).

FIG. 6F: CFTRith-172 sensitive short circuit currents observed in non-CF, uncorrected and corrected CF HBECs as a function of editing (n=3 donors, non-CF: n=2 donors).

FIGS. 7A and 7B: Edited CF cells cultured on ALI differentiate into a sheet with basal cells (Krt5⁺), ciliated (α-tubulin+) and mucus (Muc5B+) producing cells.

FIGS. 8A and 8B: Edited cells plated on pSIS membranes at a density of 10⁵ cells/cm² resulted in 50-70% confluence in four days.

FIG. 8C: Hematoxylin and eosin staining shows a monolayer of cells on pSIS membranes (scale=50 μm).

FIGS. 8D-8F: Sheets fixed on day 4 after embedding on pSIS membrane were Krt5⁺. Calcein green indicates live cells and Krt5⁺ cells are stained red. A few cells are positive for calcein green but not Krt5 (\hat{ }). Some non-viable cells were still Krt5⁺ (*). Mander's coefficients were calculated. The fraction of calcein green positive cells also positive for Krt5 was determined to be 78% for sample presented here (average=53±15% for n=4 biological replicates).

FIG. 9A: Sinus basal cells cultured in UNC media showed higher transepithelial resistances after differentiation on ALI.

FIG. 9B: Representative traces from epithelial sheets cultured in pneumacult ALI and UNC media. Sheets cultured in UNC media showed a more pronounced forskolin response.

FIG. 9C: Short circuit currents in response to forskolin were higher in sheets cultured in UNC media. The presence of absence of collagen IV coating did not make a difference.

FIG. 9D: Responses to CFTRᵢₙₕ-172 were similar between sheets cultured in UNC media and pneumacult for non-CF cells.

FIGS. 9E-9G: Sinus cells from ΔF508 homozygous patient were edited (27% allelic correction) and differentiated using pneumacult ALI and UNC media (no collagen IV coating).

FIG. 10: Embedding cells on an SIS membrane was most successful at densities greater than 50,000 cells/cm².

FIG. 11: Cells edited at the CF locus and embedded on an SIS membrane remained Krt5⁺ basal cells.

FIGS. 12A and 12B: Airway basal cells seeded on the SIS membrane are positive for stemness markers p63 and cytokeratin 14.

FIGS. 12C and 12D: Airway basal cells seeded on the SIS membrane retained their CFTR function, similar to that of basal cells cultured on Matrigel coated plates.

### DETAILED DESCRIPTION OF THE EMBODIMENTS

### I. Introduction

The discovery of the Cystic Fibrosis Transmembrane Conductance Regulator (CFTR) protein in 1989 resulted in several attempts to treat CF using gene therapy.¹⁹ These studies used various viral and non-viral strategies but failed to show significant benefits.¹⁹ The recent development of genome editing using Cas9 and other nucleases (e.g., zinc finger) has resulted in a renewed effort to correct CF-causing mutations in intestinal cells and induced pluripotent cells.⁴⁻⁶ Despite these studies, the correction of CFTR mutations in primary airway stem cells has been a persistent challenge. In general, it is difficult to adopt gene editing approaches to epithelial stem cells because they are few in number and exist in isolated locations in the submucosal glands where the presence of the mucin layer and mucus makes gene editing challenging.

The present disclosure provides an efficient, selection-free, and clinically compatible approach to generate cell-based therapies for airway diseases (e.g., CF) from autologous airway stem cells. For example, the present disclosure describes methods of using a Cas protein to correct CFTR mutations in human airway stem cells. The experiments described herein demonstrated using Cas9 and AAV6 to correct the ΔF508 mutation in the CFTR protein, which is seen in >70% of CF patients, in ex-vivo expanded human upper and lower airway cytokeratin 5+(Krt5⁺) stem cells from sinus and bronchial epithelium obtained from CF and non-CF patients undergoing endoscopic sinus surgery. The ex-vivo correction strategy overcomes several challenges associated with in vivo gene correction, such as delivery across the thick mucus barrier,⁷ immunogenicity to Cas9 in humans⁸ as well as mice⁹, and achieving high levels of homologous recombination in quiescent stem cells in vivo.

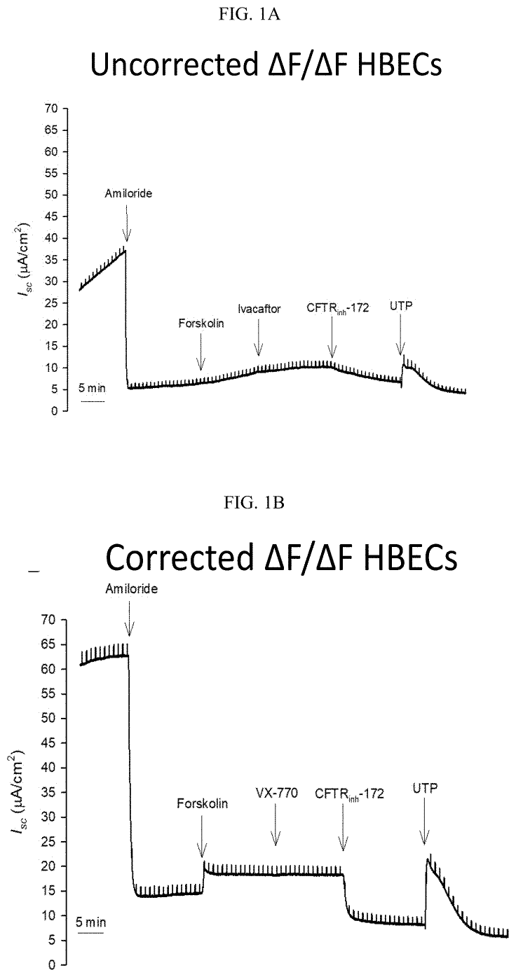

As described in the examples, the present disclosure demonstrates correction of the ΔF508 mutation in the CFTR protein in about 40% alleles in sinus and bronchial cells obtained from CF patients. Further, this correction was achieved without the use of any selection strategy. This level of correction is a 100-fold improvement over previous studies.⁴ Corrected sinus and bronchial basal cells gave rise to differentiated epithelia with ciliated and mucus producing cells. Different media and culture conditions have been reported for the culture of epithelial sheets in air-liquid interface.²⁰ The commercially available pneumacult ALI medium and ALI medium previously reported by Randell et al.²⁰ were tested. The response to CFTRᵢₙₕ-172 was similar under both conditions in both non-CF and corrected CF cells (FIGS. 1A and 1B).

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “airway stem cells” refers to undifferentiated cells, which are multipotent and capable of self-renewal, present in the airway. An airway includes an upper airway (e.g., the nasal cavities, the pharynx, and larynx) and a lower airway (e.g., trachea, bronchi (e.g., mainstem bronchus, lobar bronchus, and segmental bronchus), bronchiole, alveolar duct, and alveolus). Airway stem cells may be found near the submucosal glands (e.g., the ductal epithelia of the submucosal glands) and the basal cells of the basement membrane. As used herein, the terms “basal cell” and “basal stem cell” are used interchangeably. In some embodiments, airway stem cells express cell markers such as cytokeratin 5 (Krt5), CC10, and/or AT2. In particular embodiments, airway stem cells (e.g., stem cells in sinus and lower airway epithelia) express Krt5. An airway stem cell may be a naturally-occurring airway stem cell or a gene edited airway stem cell.

The term “gene edited airway stem cell” refers to an airway stem cell that is genetically edited or altered by nuclease-mediated genome editing (e.g., a CRISPR/Cas nuclease system) such that a heterologous nucleic acid has been introduced, in some cases, into its endogenous genomic DNA. In some embodiments, an airway stem cell is genetically edited to correct a mutation in a protein.

The term “CFTR protein” or “Cystic Fibrosis Transmembrane Conductance Regulator (CFTR) protein” refers to a membrane protein and chloride channel that is encoded by the CFTR gene. A CFTR protein may be a wild-type CFTR protein or a mutated CFTR protein. In some embodiments, a wild-type protein may be encoded by the nucleic acid sequence shown in GenBank ID NO: NM_000492.3 and have the amino acid sequence shown in SEQ ID NO: 11 below. A mutated CFTR protein may have one or more amino acid mutations shown in Table 1 (e.g., ΔF508 mutation and/or R117H) relative to a sequence of a wild-type CFTR protein (e.g., a wild-type CFTR protein having the sequence of SEQ ID NO: 11).

SEQ ID NO: 11:
MQRSPLEKASVVSKLFFSWTRPILRKGYRQRLELSDIYQIPSVDSADNLS
EKLEREWDRELASKKNPKLINALRRCFFWRFMFYGIFLYLGEVTKAVQPL
LLGRIIASYDPDNKEERSIAIYLGIGLCLLFIVRTLLLHPAIFGLHHIGM
QMRIAMFSLIYKKTLKLSSRVLDKISIGQLVSLLSNNLNKFDEGLALAHF
VWIAPLQVALLMGLIWELLQASAFCGLGFLIVLALFQAGLGRMMMKYRDQ
RAGKISERLVITSEMIENIQSVKAYCWEEAMEKMIENLRQTELKLTRKAA
YVRYFNSSAFFFSGFFVVFLSVLPYALIKGIILRKIFTTISFCIVLRMAV
TRQFPWAVQTWYDSLGAINKIQDFLQKQEYKTLEYNLTTTEVVMENVTAF
WEEGFGELFEKAKQNNNNRKTSNGDDSLFFSNFSLLGTPVLKDINFKIER
GQLLAVAGSTGAGKTSLLMVIMGELEPSEGKIKHSGRISFCSQFSWIMPG
TIKENIIFGVSYDEYRYRSVIKACQLEEDISKFAEKDNIVLGEGGITLSG
GQRARISLARAVYKDADLYLLDSPFGYLDVLTEKEIFESCVCKLMANKTR
ILVTSKMEHLKKADKILILHEGSSYFYGTFSELQNLQPDFSSKLMGCDSF
DQFSAERRNSILTETLHRFSLEGDAPVSWTETKKQSFKQTGEFGEKRKNS
ILNPINSIRKFSIVQKTPLQMNGIEEDSDEPLERRLSLVPDSEQGEAILP
RISVISTGPTLQARRRQSVLNLMTHSVNQGQNIHRKTTASTRKVSLAPQA
NLTELDIYSRRLSQETGLEISEEINEEDLKECFFDDMESIPAVTTWNTYL
RYITVHKSLIFVLIWCLVIFLAEVAASLVVLWLLGNTPLQDKGNSTHSRN
NSYAVIITSTSSYYVFYIYVGVADTLLAMGFFRGLPLVHTLITVSKILHH
KMLHSVLQAPMSTLNTLKAGGILNRFSKDIAILDDLLPLTIFDFIQLLLI
VIGAIAVVAVLQPYIFVATVPVIVAFIMLRAYFLQTSQQLKQLESEGRSP
IFTHLVTSLKGLWTLRAFGRQPYFETLFHKALNLHTANWFLYLSTLRWFQ
MRIEMIFVIFFIAVTFISILTTGEGEGRVGIILTLAMNIMSTLQWAVNSS
IDVDSLMRSVSRVFKFIDMPTEGKPTKSTKPYKNGQLSKVMHENSHVKKD
DIWPSGGQMTVKDLTAKYTEGGNAILENISFSISPGQRVGLLGRTGSGKS
TLLSAFLRLLNTEGEIQIDGVSWDSITLQQWRKAFGVIPQKVFIFSGTFR
KNLDPYEQWSDQEIWKVADEVGLRSVIEQFPGKLDFVLVDGGCVLSHGHK
QLMCLARSVLSKAKILLLDEPSAHLDPVTYQIIRRTLKQAFADCTVILCE
HRIEAMLECQQFLVIEENKVRQYDSIQKLLNERSLFRQAISPSDRVKLFP
HRNSSKCKSKPQIAALKEETEEEVQDTRL

The term “decellularized ECM membrane” refers to a membrane derived from the extracellular matrix of a tissue that underwent a decellularization process (i.e., a removal of cells from the tissue) and is thus devoid of any cellular components. A decellularized ECM membrane serves as a network or scaffold supporting the attachment and proliferation of the airway stem cells (e.g., airway stem cells expressing Krt5). In particular embodiments, a decellularized ECM membrane may be made from small intestinal submucosal (SIS) membrane (e.g., porcine SIS (pSIS) membrane).

The term “airway disease” refers a disease that affects one or more parts of a subject's airway, e.g., the upper airway (e.g., the nasal cavities, the pharynx, and larynx) and the lower airway (e.g., trachea, bronchi (e.g., mainstem bronchus, lobar bronchus, and segmental bronchus), bronchiole, alveolar duct, and alveolus). In some embodiments, an airway disease may be caused by a genetic mutation (which may cause an amino acid mutation in a protein of the subject) and/or a mutated protein. In some embodiments, an airway disease is cystic fibrosis (CF). A major cause of cystic fibrosis is a genetic mutation that causes a ΔF508 mutation in the CFTR protein.

The term “amino acid mutation” refers to a change in the amino acid sequence of a wild-type protein. An amino acid mutation may be an amino acid substitution, addition, or deletion at a specific amino acid position.

The term “gene” refers to a combination of polynucleotide elements, that when operatively linked in either a native or recombinant manner, provide some product or function. The term “gene” is to be interpreted broadly, and can encompass mRNA, cDNA, cRNA and genomic DNA forms of a gene.

The term “homology-directed repair” or “HDR” refers to a mechanism in cells to accurately and precisely repair double-strand DNA breaks using a homologous template to guide repair. The most common form of HDR is homologous recombination (HR).

The term “homologous recombination” or “HR” refers to a genetic process in which nucleotide sequences are exchanged between two similar molecules of DNA. Homologous recombination (HR) is used by cells to accurately repair harmful breaks that occur on both strands of DNA, known as double-strand breaks or other breaks that generate overhanging sequences.

The term “single guide RNA” or “sgRNA” refers to a DNA-targeting RNA containing a guide sequence that targets the Cas nuclease to the target genomic DNA and a scaffold sequence that interacts with the Cas nuclease (e.g., tracrRNA), and optionally, a donor repair template.

The term “Cas polypeptide” or “Cas nuclease” refers to a Clustered Regularly Interspaced Short Palindromic Repeats-associated polypeptide or nuclease that cleaves DNA to generate blunt ends at the double-strand break at sites specified by a 20-nucleotide guide sequence contained within a crRNA transcript. A Cas nuclease requires both a crRNA and a tracrRNA for site-specific DNA recognition and cleavage. The crRNA associates, through a region of partial complementarity, with the tracrRNA to guide the Cas nuclease to a region homologous to the crRNA in the target DNA called a “protospacer.”

The term “ribonucleoprotein complex” or “RNP complex” refers to a complex comprising an sgRNA and a Cas polypeptide.

The term “homologous donor adeno-associated viral vector” or “donor adeno-associated viral vector” refers to an adeno-associated viral particle that can express a recombinant donor template for CRISPR-based gene editing via homology-directed repair in a host cell, e.g., primary cell.

The term “recombinant donor template” refers to a nucleic acid stand, e.g., DNA strand that is the recipient strand during homologous recombination strand invasion that is initiated by the damaged DNA, in some cases, resulting from a double-stranded break. The donor polynucleotide serves as template material to direct the repair of the damaged DNA region.

The term “percent (%) sequence identity” refers to the percentage of amino acid or nucleic acid residues of a candidate sequence that are identical to the amino acid or nucleic acid residues of a reference sequence after aligning the sequences and introducing gaps, if necessary, to achieve the maximum percent identity (i.e., gaps can be introduced in one or both of the candidate and reference sequences for optimal alignment). In some embodiments, percent sequence identity can be any integer from 50% to 100%. In some embodiments, a sequence is substantially identical to a reference sequence if the sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined using the methods described herein; preferably BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Alignment for purposes of determining percent sequence identity can be achieved in various ways that are within the skill in the art, for instance, using publicly available computer software such as BLAST, ALIGN, or Megalign (DNASTAR) software. The BLAST and BLAST 2.0 algorithms are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. Those skilled in the art can determine appropriate parameters for measuring alignment, including any algorithms needed to achieve maximal alignment over the full length of the sequences being compared. In some embodiments, the percent amino acid or nucleic acid sequence identity of a given candidate sequence to, with, or against a given reference sequence (which can alternatively be phrased as a given candidate sequence that has or includes a certain percent amino acid or nucleic acid sequence identity to, with, or against a given reference sequence) is calculated as follows:

100×(fraction of A/B)

where A is the number of amino acid or nucleic acid residues scored as identical in the alignment of the candidate sequence and the reference sequence, and where B is the total number of amino acid or nucleic acid residues in the reference sequence. In some embodiments where the length of the candidate sequence does not equal to the length of the reference sequence, the percent amino acid or nucleic acid sequence identity of the candidate sequence to the reference sequence would not equal to the percent amino acid or nucleic acid sequence identity of the reference sequence to the candidate sequence.

In particular embodiments, a reference sequence aligned for comparison with a candidate sequence may show that the candidate sequence exhibits from 50% to 100% identity across the full length of the candidate sequence or a selected portion of contiguous amino acid or nucleic acid residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid or nucleic acid residue as the corresponding position in the reference sequence, then the molecules are identical at that position.

The term “homologous” refers to two or more amino acid sequences when they are derived, naturally or artificially, from a common ancestral protein or amino acid sequence. Similarly, nucleotide sequences are homologous when they are derived, naturally or artificially, from a common ancestral nucleic acid.

The term “administering or “administration” refers to the process by which agents, compositions, dosage forms and/or combinations disclosed herein are delivered to a subject for treatment or prophylactic purposes. Compositions, dosage forms and/or combinations disclosed herein are administered in accordance with good medical practices taking into account the subject's clinical condition, the site and method of administration, dosage, subject age, sex, body weight, and other factors known to the physician. For example, the terms “administering” or “administration” include providing, giving, grafting, transplanting, dosing, and/or prescribing agents, compositions, dosage forms and/or combinations disclosed herein by a clinician or other clinical professional.

The term “treating” refers to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The terms “culture,” “culturing,” “grow,” “growing,” “maintain,” “maintaining,” “expand,” “expanding,” etc., when referring to cell culture itself or the process of culturing, can be used interchangeably to mean that a cell (e.g., an airway stem cell) is maintained outside its normal environment under controlled conditions, e.g., under conditions suitable for survival. Cultured cells are allowed to survive, and culturing can result in cell growth, stasis, differentiation, or division. The term does not imply that all cells in the culture survive, grow, or divide, as some may naturally die or senesce. Cells are typically cultured in media, which can be changed during the course of the culture.

The terms “subject,” “patient,” and “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

Unless defined otherwise, all technical and scientific terms used herein have the same meanings as commonly understood by one of ordinary skill in the art to which this technology belongs. Although exemplary methods, devices and materials are described herein, any methods and materials similar or equivalent to those expressly described herein can be used in the practice or testing of the present technology. For example, the reagents described herein are merely exemplary and that equivalents of such are known in the art. The practice of the present technology can employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR I: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Miller and Calos eds. (1987) Gene Transfer Vectors for Mammalian Cells (Cold Spring Harbor Laboratory); and Makrides ed. (2003) Gene Transfer and Expression in Mammalian Cells (Cold Spring Harbor Laboratory).

### III. Compositions

The present disclosure provides a composition for airway tissue regeneration that includes an airway stem cell and a bioscaffold (e.g., a decellularized extracellular matrix (ECM) membrane), wherein the airway stem cell expresses cytokeratin 5 (Krt5) and is embedded in the bioscaffold (e.g., the decellularized ECM membrane). The composition may be used to treat a subject having an airway disease (e.g., cystic fibrosis (CF)).

Airway stem cells in the composition are undifferentiated cells, which are multipotent and capable of self-renewal, present in the upper airway (e.g., the nasal cavities, the pharynx, and larynx) and/or a lower airway (e.g., trachea, bronchi (e.g., mainstem bronchus, lobar bronchus, and segmental bronchus), bronchiole, alveolar duct, and alveolus). Airway stem cells may be found near the submucosal glands (e.g., the ductal epithelia of the submucosal glands) and the basal cells of the basement membrane. In some embodiments, airway stem cells express cell markers such as cytokeratin 5 (Krt5), CC10, and/or AT2. In particular embodiments, airway stem cells (e.g., upper airway and lower airway stem cells; stem cells in sinus and lower airway epithelia) express Krt5. In some embodiments, an airway stem cell may be an upper airway stem cell (e.g., a nasal stem cell), such as an upper airway basal stem cell (e.g., a sinus basal stem cell). In some embodiments, an airway stem cell may be a lower airway stem cell, such as a bronchial stem cell (e.g., a human bronchial epithelial cell (HBEC)). An airway stem cell may be a naturally-occurring airway stem cell or a gene edited airway stem cell.

In some embodiments, the airway stem cell in the composition may be a naturally-occurring airway stem cell that expresses wild-type proteins. In some embodiments, the airway stem cell in the composition may be a naturally-occurring airway stem cell that expresses a wild-type Cystic Fibrosis Transmembrane Conductance Regulator (CFTR) protein. In some embodiments, the airway stem cell in the composition may be a gene edited airway stem cell. For example, an airway stem cell may be isolated from a subject having an airway disease (e.g., CF) that is caused by an amino acid mutation in a protein. Once isolated, the airway stem cell may be gene edited (i.e., gene edited using a CRISPR/Cas nuclease system) to correct the amino acid mutation, then expanded ex vivo (i.e., regenerated by embedding in a decellularized ECM membrane) before being reintroduced into the subject having the airway disease (e.g., CF). Several mutated forms of the CFTR protein have been observed in patients and these mutations can occur throughout the coding region of the protein. Table 1 below lists mutations in the CFTR protein with an allelic frequency of at least 0.8%.

TABLE 1
Mutations in CFTR Protein

| | | | | | Allele frequency in |
|---|---|---|---|---|---|
| Variant cDNA name | Variant | Variant | | # alleles | CFTR (of 142,036 |
| (ordered 5′ to 3′) | protein name | legacy name | SNP | in CFTR | identified variants)* |
| c.350G>A | p.Arg117His | R117H | rs78655421 | 1854 | 0.01305 |
| c.489+1G>T | No protein name | 621+1G->T | rs78756941 | 1323 | 0.00931 |
| c.1521_1523delCTT | p.Phe508del | F508del | rs113993960 | 99061 | 0.69744 |
| c.1624G>T | p.Gly542X | G542X | rs113993959 | 3610 | 0.02542 |
| c.1652G>A | p.Gly551Asp | G551D | rs75527207 | 2986 | 0.02102 |
| c.1657C>T | p.Arg553X | R553X | rs74597325 | 1323 | 0.00931 |
| c.3718-2477C>T | No protein name | 3849+10kbC->T | rs75039782 | 1158 | 0.00815 |
| c.3846G>A | p.Trp1282X | W1282X | rs77010898 | 1726 | 0.01215 |
| c.3909C>G | p.Asn1303Lys | N1303K | rs80034486 | 2246 | 0.01581 |

A mutated CFTR protein having a deletion of phenylalanine at position 508 (ΔF508 mutation) is found in subjects having CF. A mutated CFTR protein having an amino acid substitution of R117H is also found in subjects having CF. In some embodiments, for example, an airway stem cell isolated from a subject having CF may be gene edited to correct the ΔF508 mutation and/or the amino acid substitution of R117H. In other embodiments, other mutation specific platforms or the complete coding sequence of the CFTR protein may be inserted into the CFTR locus. Once the amino acid mutation (e.g., ΔF508 mutation and/or the amino acid substitution of R117H; mutations listed in Table 1) is corrected, the gene edited airway stem cells, now expressing a wild-type CFTR (i.e., a wild-type CFTR protein having phenylalanine at position 508 and arginine at position 117; a wild-type CFTR protein having the sequence of SEQ ID NO: 11), or the complete CFTR protein coding sequence in exon 1, or other variations resulting in the wild-type CFTR protein, may be embedded in a decellularized ECM membrane to form the composition for airway tissue regeneration.

In some embodiments, airway stem cells embedded in the bioscaffold (e.g., the decellularized ECM membrane) may give rise to differentiated airway ciliated cells (e.g., airway ciliated cells expressing acetylated alpha tubulin) and/or airway mucus producing cells (e.g., airway mucus producing cells expressing MUC5AC). Thus, the composition described herein may further include airway ciliated cells and/or airway mucus producing cells.

In some embodiments, the bioscaffold in a composition described herein is a decellularized ECM membrane. The decellularized ECM membrane in the composition serves as a network or scaffold supporting the attachment and proliferation of the airway stem cells (e.g., airway stem cells expressing Krt5). The decellularized ECM membrane may mimic the microenvironment of the airway (e.g., nasal cavity or bronchi). In some embodiments, airway stem cells retain Krt5 expression after being embedded and grown in the decellularized ECM membrane. A decellularized ECM membrane may be derived from a tissue source (e.g., a mammalian tissue source) selected from the group consisting of intestine tissue, pancreas tissue, liver tissue, lung tissue, trachea tissue, esophagus tissue, kidney tissue, bladder tissue, skin tissue, heart tissue, brain tissue, placenta tissue, and umbilical cord tissue. In particular embodiments, a decellularized ECM membrane may be made from small intestinal submucosal (SIS) membrane (e.g., porcine SIS (pSIS) membrane).

### IV. Methods for Airway Tissue Regeneration

The present disclosure provides a method for airway tissue regeneration that includes: (a) inducing a stable gene modification of a target nucleic acid encoding a mutated protein in an airway stem cell via homologous recombination by introducing into the airway stem cell: (1) a single guide RNA (sgRNA) comprising a first nucleotide sequence that is complementary to the target nucleic acid, and a second nucleotide sequence that interacts with a CRISPR-associated protein (Cas) polypeptide; (2) a Cas polypeptide, an mRNA encoding a Cas polypeptide, and/or a recombinant expression vector comprising a nucleotide sequence encoding a Cas polypeptide, wherein the sgRNA guides the Cas polypeptide to the target nucleic acid; and (3) a homologous donor adeno-associated viral (AAV) vector comprising a recombinant donor template comprising two nucleotide sequences comprising two non-overlapping, homologous portions of the target nucleic acid, wherein the nucleotide sequences are located at the 5′ and 3′ ends of a nucleotide sequence corresponding to the target nucleic acid to undergo homologous recombination; (b) embedding the airway stem cell in a bioscaffold (e.g., a decellularized extracellular matrix (ECM) membrane); (c) culturing the airway stem cell embedded in the bioscaffold (e.g., the decellularized ECM membrane), wherein the airway stem cell expresses Krt5. As demonstrated in the examples, the gene edited cells embedded successfully on an FDA approved porcine small intestinal submucosal (pSIS) membrane, which was previously shown to improve re-mucosalization after sinus surgery.

In some embodiments of the method, in step (a)(3), the homologous donor AAV vector may be selected from a wild-type AAV serotype 1 (AAV1), wild-type AAV serotype 2 (AAV2), wild-type AAV serotype 3 (AAV3), wild-type AAV serotype 4 (AAV4), wild-type AAV serotype 5 (AAV5), wild-type AAV serotype 6 (AAV6), wild-type AAV serotype 7 (AAV7), wild-type AAV serotype 8 (AAV8), wild-type AAV serotype 9 (AAV9), wild-type AAV serotype 10 (AAV10), wild-type AAV serotype 11 (AAV11), wild-type AAV serotype 12 (AAV12), a variant thereof, and any shuffled chimera thereof. In certain embodiments, the homologous donor AAV vector is a wild-type AAV6 or an AAV6 variant having at least 95% (e.g., 97%, 99%, or 100%) sequence identity to wild-type AAV6.

In some embodiments, the airway stem cell includes a population of airway stem cells. The stable gene modification of the target nucleic acid may be induced in greater than about 70% (e.g., greater than about 75%, 80%, 85%, 90%. 95%, or 97%) of the population of airway stem cells.

The sgRNA and the Cas polypeptide may be incubated together first to form a ribonucleoprotein (RNP) complex prior to introducing (i.e., via electroporation) into the airway stem cell. Subsequently, the RNP complex and the homologous donor AAV vector may be concomitantly introduced into the airway stem cell or sequentially introduced into the airway stem cell (i.e., the RNP complex is introduced into the airway stem cell before the homologous donor AAV vector).

In some embodiments, in step (a) of the method, the mutated protein may be a mutated CFTR protein having one or more amino acid mutations (e.g., mutations listed in Table 1). A mutated CFTR protein, which is a major cause of CF, may have a ΔF508 mutation and/or other amino acid mutations (e.g., amino acid substitution R117H; mutations listed in Table 1). A CRISPR/Cas nuclease system may be used to gene edit the nucleic acid encoding the mutated CFTR protein to correct the ΔF508 mutation and/or the other amino acid mutations (e.g., amino acid substitution R117H; mutations listed in Table 1), such that the modified nucleic acid encodes a wild-type CFTR protein (e.g., a wild-type CFTR protein having the sequence of SEQ ID NO: 11).

In some embodiments of the method, the airway stem cell embedded in the bioscaffold (e.g., the decellularized ECM membrane) differentiates into airway ciliated cells (e.g., airway ciliated cells expressing acetylated alpha tubulin) and/or airway mucus producing cells (e.g., airway mucus producing cells expressing MUC5AC).

The present disclosure also provides ex vivo regenerated airway stem cells (e.g., airway stem cells expressing Krt5, upper airway stem cells (e.g., nasal stem cells), such as upper airway basal stem cells (e.g., sinus basal stem cells), or lower airway stem cells, such as bronchial stem cells (e.g., human bronchial epithelial cells (HBECs))) produced by the methods described herein. Other airway cells may also be expanded or regenerated using the methods described herein, such as Type-I cells and Type-II cells.

Airway stem cells (e.g., airway stem cells expressing Krt5) may be cultured by embedding the cells in a bioscaffold, such as a decellularized extracellular cell matrix (ECM) membrane. A bioscaffold refers to a substrate or matrix on which cells can grow and may be derived from or made from natural or synthetic tissues or cells or other natural or synthetic materials. In some embodiments, a bioscaffold may be derived from, made from, and/or comprises natural or synthetic materials such as extracellular matrix, collagen Type I, collagen Type IV, fibronectin, polycarbonate, and polystyrene. In some embodiments, a bioscaffold may include a decellularized extracellular matrix (ECM) membrane. A bioscaffold may be used for tissue or cell engineering and/or ex vivo expansion or regeneration. A bioscaffold may be in the form of a membrane, a matrix, a microbead, or a gel (e.g., a hydrogel), and/or a combination thereof. A bioscaffold can be made out of materials that have the physical or mechanical attributes required for grafting or implantation. In some embodiments, the bioscaffold is made of a semi-permeable material which may include collagen (e.g., collagen Type-I, collagen Type-IV), which may be cross-linked or uncross-linked. The bioscaffold may also include polypeptides or proteins obtained from natural sources or by synthesis, such as hyaluronic acid, small intestine submucosa (SIS), peritoneum, pericardium, polylactic acids and related acids, blood (i.e., which is a circulating tissue including a fluid portion (plasma) with suspended formed elements (red blood cells, white blood cells, platelets)), or other materials that are bioresorbable (e.g., bioabsorbable polymers, such as elastin, fibrin, laminin, and fibronectin).

A bioscaffold may have one or several surfaces, such as a porous surface, a dense surface, or a combination of both. The bioscaffold may also include semi-permeable, impermeable, or fully permeable surfaces. The bioscaffold may be autologous or allogeneic. A bioscaffold may be a solid, semi-solid, gel, or gel-like scaffold characterized by being able to hold a stable form for a period of time to enable the adherence and/or growth of cells thereon, both before grafting and after grafting, and to provide a system similar to the natural environment of the cells to optimize cell growth. Some examples of bioscaffolds include, but are not limited to, Vitrogen™, a collagen-containing solution which gels to form a cell-populated matrix, and the connective-tissue scaffolds described in US Patent Publication No. 20040267362). A bioscaffold can be cut or formed into any regular or irregular shape. In some embodiments, the bioscaffold can be cut to correspond to the shape of the area where it is to be grafted. The bioscaffold can be flat, round, and/or cylindrical in shape. In some embodiments, a bioscaffold may include type I/III collagen (e.g., collagen Type-I). In some embodiments, a bioscaffold may include small intestinal submucosa.

In some embodiments, a bioscaffold is a decellularized ECM membrane. A decelluarlized ECM membrane may include collagen (e.g., collagen Type-I), elastic fibers, glycosoaminoglycans, proteoglycans, and adhesive glycoproteins. The decellularized ECM membrane serves as a network or scaffold supporting the attachment and proliferation of the airway stem cells (e.g., airway stem cells expressing Krt5). The decellularized ECM membrane may mimic the microenvironment of the airway (e.g., nasal cavity or bronchi). In some embodiments, airway stem cells retain Krt5 expression after being embedded and grown in the decellularized ECM membrane.

A decellularized ECM membrane may be derived from a mammalian tissue source, such as a tissue from human, monkey, pig, cow, sheep, horse, goat, mouse, and rat. The tissue source from which to make the decellularized ECM membrane may be from any organ or tissue of a mammal, including without limitation, intestine tissue, pancreas tissue, liver tissue, lung tissue, trachea tissue, esophagus tissue, kidney tissue, bladder tissue, skin tissue, heart tissue, brain tissue, placenta tissue, and umbilical cord tissue. Further, the decellularized ECM membrane may include any tissue obtained from an organ, including, for example and without limitation, submucosa, epithelial basement membrane, and tunica propria. In some embodiments, the decellularized ECM membrane may be made from small intestinal submucosal (SIS) membrane. In particular embodiments, the decellularized ECM membrane may be made from porcine SIS (pSIS) membrane.

Methods of preparing decellularized ECM membranes are known in the art. Generation of decellularized ECM membranes from tissues generally involves subjecting the tissues to enzymatic cellular digestion (e.g., using trypsin), hypotonic, hypertonic, and/or low ionic strength buffers, detergent, and chemical digestion (e.g., using SDS, Triton-X-100, ammonium hydroxide, and/or peracetic acid), and non-micellar amphipathic molecules such as polyethylene glycol (PEG). Examples of methods of preparing decellularized ECM membranes are described in, e.g., U.S. Patent Application Publication Nos. 2004/0076657, 2003/0014126, 20050191281, 2005/0256588, and U.S. Pat. Nos. 6,933,103, 6,743,574, 6,734,018, 5,855,620, each of which is incorporated herein by reference in its entirety. Commercially available decellularized ECM membrane preparations can also be used. Commercially available preparations for decellularized ECM membranes from SIS membranes include, but are not limited to, Surgisis™, Surgisis-ES™, Stratasis™, and Stratasis-ES™ (Cook Urological Inc.; Indianapolis, Ind.) and GraftPatch™ (Organogenesis Inc.; Canton Mass.). Commercially available preparations for decellularized ECM membranes from dermis include, but are not limited to Pelvicol™ (sold as Permacol™ in Europe; Bard, Covington, Ga.), Repliform™ (Microvasive; Boston, Mass.), and Alloderm™ (LifeCell; Branchburg, N.J.). Commercially available preparations for decellularized ECM membranes from urinary bladder include, but are not limited to, UBM (Acell Corporation; Jessup, Md.).

A decellularized ECM membrane can have suitable viscoelasticity and flow behavior for grafting or injecting to the desired area (e.g., airway (e.g., nasal cavity or bronchi)) for clinical treatment. For example, and not by way of limitation, the viscosity of a decellularized ECM membrane can be in a range between 100 to 400 Pa·s (e.g., between 100 to 400 Pa·s, between 100 to 380 Pa·s, between 100 to 360 Pa·s, between 100 to 340 Pa·s, between 100 to 320 Pa·s, between 100 to 300 Pa·s, between 100 to 280 Pa·s, between 100 to 260 Pa·s, between 100 to 240 Pa·s, between 100 to 220 Pa·s, between 100 to 200 Pa·s, between 100 to 180 Pa·s, between 100 to 160 Pa·s, between 100 to 140 Pa·s, between 100 to 120 Pa·s, between 120 to 400 Pa·s, between 140 to 400 Pa·s, between 160 to 400 Pa·s, between 180 to 400 Pa·s, between 200 to 400 Pa·s, between 220 to 400 Pa·s, between 240 to 400 Pa·s, between 260 to 400 Pa·s, between 280 to 400 Pa·s, between 300 to 400 Pa·s, between 320 to 400 Pa·s, between 340 to 400 Pa·s, between 360 to 400 Pa·s, or between 380 to 400 Pa·s). In some embodiments, a decellularized ECM membrane can have a suitable thickness for grafting or injecting to the desired area (e.g., airway (e.g., nasal cavity or bronchi)) for clinical treatment. For example, and not by way of limitation, the thickness of a decellularized ECM membrane can be in a range between 100 to about 2000 μm (e.g., between 100 to about 1500 μm, between 100 to about 1000 μm, between 100 to about 900 μm, between 100 to about 800 μm, between 100 to about 700 μm, between 100 to about 600 μm, between 100 to about 500 μm, between 100 to about 400 μm, between 100 to about 300 μm, between 100 to about 200 μm, between 200 to about 2000, between 300 to about 2000 μm, between 400 to about 2000 μm, between 500 to about 2000 μm, between 600 to about 2000 μm, between 700 to about 2000 μm, between 800 to about 2000 μm, between 900 to about 2000 μm, between 1000 to about 2000 μm, or between 1500 to about 2000 μm). In some embodiments, a decellularized ECM membrane can contain components that are present in tissue from which it was derived. In certain embodiments, the decellularized ECM membrane can contain components that are present in airway tissue (e.g., nasal mucosal tissue or bronchial mucosal tissue) to mimic the characteristics of the airway tissue and its organization and function. For example, and not by way of limitation, the decellularized ECM membrane can include collagen (e.g., collagen Type-I), glycosaminoglycan, laminin, elastin, non-collagenous protein and the like.

Techniques and methods of culturing cells in a bioscaffold (e.g., a decellularized ECM membrane) for grafting purposes are known in the art. An optimal plating density to achieve a certain percentage of coverage in a certain period of time may be determined by a skilled artisan. As described in the examples, a plating density of greater than 50,000 cells/cm² (e.g., about 100,000 cells/cm²) was used to achieve 50-70% coverage in four days. Depending on the number of days before the expanded cells are used for grafting, the plating density may be adjusted accordingly to achieve the desired number of cells and the percentage of coverage in the decellularized ECM membrane for grafting. In some embodiments, a plating density of between 10,000 to 1,000,000 cells/cm² (e.g., between 10,000 to 900,000 cells/cm², between 10,000 to 800,000 cells/cm², between 10,000 to 700,000 cells/cm², between 10,000 to 600,000 cells/cm², between 10,000 to 500,000 cells/cm², between 10,000 to 400,000 cells/cm², between 10,000 to 300,000 cells/cm², between 10,000 to 200,000 cells/cm², between 10,000 to 100,000 cells/cm², between 10,000 to 90,000 cells/cm², between 10,000 to 80,000 cells/cm², between 10,000 to 70,000 cells/cm², between 10,000 to 60,000 cells/cm², between 10,000 to 50,000 cells/cm², between 10,000 to 40,000 cells/cm², between 10,000 to 30,000 cells/cm², between 10,000 to 20,000 cells/cm², between 10,000 to 15,000 cells/cm², between 20,000 to 1,000,000 cells/cm², between 30,000 to 1,000,000 cells/cm², between 40,000 to 1,000,000 cells/cm², between 50,000 to 1,000,000 cells/cm², between 60,000 to 1,000,000 cells/cm², between 70,000 to 1,000,000 cells/cm², between 80,000 to 1,000,000 cells/cm², between 90,000 to 1,000,000 cells/cm², between 100,000 to 1,000,000 cells/cm², between 200,000 to 1,000,000 cells/cm², between 300,000 to 1,000,000 cells/cm², between 400,000 to 1,000,000 cells/cm², between 500,000 to 1,000,000 cells/cm², between 600,000 to 1,000,000 cells/cm², between 700,000 to 1,000,000 cells/cm², between 800,000 to 1,000,000 cells/cm², or between 900,000 to 1,000,000 cells/cm²) may be used.

A cell culture medium to support the growth of airway stem cells in a bioscaffold (e.g., a decellularized ECM membrane) may be a mammalian cell culture medium. A cell culture medium may include, without limitation, salts (e.g., zinc, iron, magnesium, calcium, and potassium), vitamins (e.g., vitamin B6 (pyridoxine), vitamin B12 (cyanocobalamin), vitamin K (biotin), vitamin C (ascorbic acid), vitamin B2 (riboflavin), vitamin B1 (thiamine), vitamin B5 (D calcium pentothenate), and vitamin B9 (folic acid)), amino acids, buffering agents (e.g., NaHCO₃, CaCl₂, MgSO₄, NaH₂PO₄, beta-glycerol-phosphate, bicarbonate, sodium pyruvate, HEPES, and MOPS), carbohydrates (e.g., mannose, fructose, galactose, maltose, and glucose), and growth factors (e.g., EGF, BMPs, EPOs, and ILs). Examples of cell culture media include, for example, Iscove's Modified Dulbecco's Medium, RPMI 1640, Dulbecco's Modified essential Medium (DMEM), Minimal Essential Medium-alpha (MEM-alpha), MCDB media, and Ham's F12. In some embodiments, airway stem cells are maintained and expanded in serum-free conditions. Alternative media, supplements and growth factors and/or alternative concentrations can readily be determined by the skilled person and are extensively described in the literature.

### V. Methods of Treatment

A subject having an airway disease that is caused by a mutated protein may be treated by grafting a composition including an airway stem cell and a bioscaffold (e.g., a decellularized ECM membrane (e.g., a porcine small intestinal submucosal (pSIS) membrane)), wherein the airway stem cell expresses Krt5 and a corresponding wild-type protein of the mutated protein, and wherein the airway stem cell is embedded in the bioscaffold (e.g., the decellularized ECM membrane). In some embodiments, the airway disease is cystic fibrosis (CF). In a majority of cystic fibrosis (CF) patients, the CF is caused by a mutated CFTR protein having a ΔF508 mutation. Multiple other mutations (e.g., R117H; mutations listed in Table 1) scattered throughout the CFTR protein sequence have also been reported. In other embodiments, the CF is caused by a mutated CFTR protein having an amino acid substitution R117H. In some embodiments, prior to being treated, an airway stem cell from the subject having the airway disease (e.g., CF) caused by a mutated protein (e.g., a mutated CFTR protein; a mutated CFTR protein having one or more mutations listed in Table 1) may be isolated and gene edited (i.e., via CRISPR/Cas nuclease system) to correct the mutated protein. Once the airway stem cell is gene edited to express the corresponding wild-type protein (e.g., a wild-type CFTR protein; a wild-type CFTR protein having the sequence of SEQ ID NO: 11) of the mutated protein, the gene edited airway stem cell may be embedded and cultured for grafting purposes in a bioscaffold (e.g., a decellularized ECM membrane).

Airway diseases that are caused by a mutated protein include, but are not limited to, cystic fibrosis, chronic bronchitis, ciliary dyskinesia, bronchiectasis, chronic occlusive pulmonary disease (COPD), and diffuse panbronchiolitis.

As described in the examples, restoration of CFTR function in ΔF508 compound heterozygous as well as homozygous samples was observed using the methods disclosed herein. Corrected Krt5⁺ sinus stem cells from CF patients differentiated in air-liquid interface and showed 8-63% restoration of CFTR Cl⁻ currents relative to non-CF cultures subject to the same assay. In contrast, corrected Krt5⁺ bronchial stem cells showed 28-110% restoration of CFTR Cl⁻ currents relative to non-CF bronchial cultures subject to the same assay. A higher level of allelic correction (about 2-fold higher) is necessary to restore CFTR function in compound heterozygous samples. For the same level of correction, CFTR function appears to be better for bronchial than sinus cells. Since the samples were not from matched donors, it is unclear if the differences are due to underlying biology of these cells or due to differences in the genetic background. Previous studies have attempted to identify the level of correction necessary to restore normal Cl⁻ transport by co-culturing non-CF or corrected CF cells and CF cells in ALI cell media. These studies have reported that 10-50% normal cells are sufficient to restore WT level Cl⁻ transport.^{21,22} The minimum amount of gene correction in the endogenous CFTR locus that can restore CFTR function has not been reported before. In the present study, homozygous samples showed the best response after correction. Compound heterozygous samples with 20% correction showed 5-10% CFTR function relative to non-CF controls and samples with 40% correction showed about 20-30% CFTR function. The results indicate that >20% allelic correction in homozygous and >40% correction in compound heterozygous samples restores 30-60% CFTR function relative to non-CF controls.

CFTR function has been reported to vary logarithmically in organ outputs measured in vivo (e.g., sweat chloride) and has been shown to be rate-limiting at low levels of CFTR expression.²³ Thus, even a low level of CFTR function may provide significant clinical benefit. For example, patients homozygous for the mutation R117H have been reported to be completely free of any respiratory or pancreatic symptoms and only present with infertility or mildly increased sweat chloride.²⁴ R117H and other class IV mutations are associated with significantly lower mortality compared to class II mutations such as ΔF508.²⁵ Patch clamp and apical conductance measurements on cells expressing exogenous R117H-CFTR showed as little as 15% Cl⁻ conductance relative to cells expressing wild-type CFTR.²⁶ By way of contrast, Wine et al. estimated <2% CFTR expression relative to WT in patients with R117H mutations.²³ Thus, the 25-60% improvement in CFTR function seen in the present study could provide a meaningful clinical benefit to patients if achieved in vivo.

Transplantation of airway stem cells into the lower airways has been reported in animal models but further optimization is necessary for clinical use.²⁷ The experiments focused on sinus basal cells since the ease of access to sinus tissue may help clinical translation. Secondly, chronic sinusitis is an unmet medical need that affects CF patients and it has been shown that the sinus in CF patients acts as a reservoir for drug resistant pathogens that cause chronic lung infections.²⁸ The area of one maxillary sinus has been estimated to be about 13 cm².²⁹ Since the results show an optimal cell density of 10⁵ cells/cm² on the pSIS membrane, 1.3 million corrected cells may be sufficient to cover one maxillary sinus completely. The corrected cells may have the ability to engraft and produce a functional epithelium.

A composition including an airway stem cell (e.g., an airway stem cell expressing Krt5) and a bioscaffold (e.g., a decellularized ECM membrane) may be administered to a subject having an airway disease (e.g., CF). In some embodiments, the composition may be grafted or injected into the airway disease site (e.g., nasal cavity or bronchi). The composition may be applied as a patch or graft overlying the airway disease site (e.g., nasal cavity or bronchi). In certain embodiments, the composition may be administered in a range of between 1 to 100 mg/cm² (e.g., between 1 to 90 mg/cm², between 1 to 80 mg/cm², between 1 to 70 mg/cm², between 1 to 60 mg/cm², between 1 to 50 mg/cm², between 1 to 40 mg/cm², between 1 to 30 mg/cm², between 1 to 20 mg/cm², between 1 to 10 mg/cm², between 1 to 5 mg/cm², between 5 to 100 mg/cm², between 10 to 100 mg/cm², between 20 to 100 mg/cm², between 30 to 100 mg/cm², between 40 to 100 mg/cm², between 50 to 100 mg/cm², between 60 to 100 mg/cm², between 70 to 100 mg/cm², between 80 to 100 mg/cm², between 90 to 100 mg/cm², or between 95 to 100 mg/cm²) of injured airway tissue.

Airway stem cells used in methods of treating an airway disease (e.g., CF) in a subject may express Krt5. Airway stem cells used in methods of treating an airway disease (e.g., CF) in a subject may be upper airway stem cells (e.g., upper airway basal stem cells), such as nasal stem cells (e.g., sinus basal stem cells). Airway stem cells used in methods of treating an airway disease (e.g., CF) in a subject may be lower airway stem cells, such as bronchial stem cells (e.g., human bronchial epithelial cells (HBECs)).

### VI. Nuclease-Mediated Genome Editing

In some embodiments of the present disclosure, a DNA nuclease such as an engineered (e.g., programmable or targetable) DNA nuclease may be used to induce genome editing of a target nucleic acid sequence. In particular, a target nucleic acid sequence may encode a mutated protein in a subject having an airway disease (e.g., CF). For example, a mutated Cystic Fibrosis Transmembrane Conductance Regulator (CFTR) protein having a deletion of phenylalanine at position 508 (ΔF508 mutation) affects >70% of subjects having CF. A DNA nuclease may be used to gene edit a target nucleic acid sequence encoding a mutated CFTR protein having ΔF508 mutation to correct the mutation such that the corrected sequence encodes a wild-type CFTR protein having phenylalanine at position 508 (e.g., a wild-type protein having the sequence of SEQ ID NO: 11). In other embodiments, subjects having CF may have a mutated CFTR protein having other amino acid mutations (i.e., amino acid mutations anywhere in the coding sequence; one or more mutations listed in Table 1 (e.g., amino acid substitution R117H)). A DNA nuclease may be used to gene edit a target nucleic acid sequence encoding a mutated CFTR protein having other amino acid mutations (e.g., amino acid substitution R117H; one or more mutations listed in Table 1) to correct the mutations such that the corrected sequence encode a wild-type CFTR protein (e.g., a wild-type CFTR protein having the sequence of SEQ ID NO: 11). In further embodiments, a DNA nuclease may be used to gene edit a target nucleic acid sequence encoding a mutated CFTR protein having ΔF508 mutation and amino acid substitution R117H to correct the mutations such that the corrected sequence encode a wild-type CFTR protein having phenylalanine at position 508 and arginine at position 117 (e.g., a wild-type CFTR protein having the seeuqnce of SEQ ID NO: 11). Any suitable DNA nuclease can be used including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In particular embodiments, CRISPR-associated protein (Cas) nucleases may be used to gene edit a mutated protein (e.g., a mutated CFTR protein) in a subject having an airway disease (e.g., CF).

In some embodiments, a nucleotide sequence encoding the DNA nuclease is present in a recombinant expression vector. In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell. For example, useful expression vectors containing a nucleotide sequence encoding a Cas9 polypeptide are commercially available from, e.g., Addgene, Life Technologies, Sigma-Aldrich, and Origene.

Depending on the target cell/expression system used, any of a number of transcription and translation control elements, including promoter, transcription enhancers, transcription terminators, and the like, may be used in the expression vector. Useful promoters can be derived from viruses, or any organism, e.g., prokaryotic or eukaryotic organisms. Suitable promoters include, but are not limited to, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc.

In other embodiments, a nucleotide sequence encoding the DNA nuclease is present as an RNA (e.g., mRNA). The RNA can be produced by any method known to one of ordinary skill in the art. As non-limiting examples, the RNA can be chemically synthesized or in vitro transcribed. In certain embodiments, the RNA comprises an mRNA encoding a Cas nuclease such as a Cas9 polypeptide or a variant thereof. For example, the Cas9 mRNA can be generated through in vitro transcription of a template DNA sequence such as a linearized plasmid containing a Cas9 open reading frame (ORF). The Cas9 ORF can be codon optimized for expression in mammalian systems. In some instances, the Cas9 mRNA encodes a Cas9 polypeptide with an N- and/or C-terminal nuclear localization signal (NLS). In other instances, the Cas9 mRNA encodes a C-terminal HA epitope tag. In yet other instances, the Cas9 mRNA is capped, polyadenylated, and/or modified with 5-methylcytidine. Cas9 mRNA is commercially available from, e.g., TriLink BioTechnologies, Sigma-Aldrich, and Thermo Fisher Scientific.

In yet other embodiments, the DNA nuclease is present as a polypeptide. The polypeptide can be produced by any method known to one of ordinary skill in the art. As non-limiting examples, the polypeptide can be chemically synthesized or in vitro translated. In certain embodiments, the polypeptide comprises a Cas protein such as a Cas9 protein or a variant thereof. For example, the Cas9 protein can be generated through in vitro translation of a Cas9 mRNA described herein. In some instances, the Cas protein such as a Cas9 protein or a variant thereof can be complexed with a single guide RNA (sgRNA) such as a modified sgRNA to form a ribonucleoprotein (RNP). Cas9 protein is commercially available from, e.g., PNA Bio (Thousand Oaks, Calif., USA) and Life Technologies (Carlsbad, Calif., USA).

CRISPR/Cas System

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease can require both the crRNA and the tracrRNA for site-specific DNA recognition and cleavage. This system has now been engineered such that the crRNA and tracrRNA can be combined into one molecule (the “single guide RNA” or “sgRNA”), and the crRNA equivalent portion of the single guide RNA can be engineered to guide the Cas (e.g., Cas9) nuclease to target any desired sequence (see, e.g., Jinek et al. (2012) Science 337:816-821; Jinek et al. (2013) eLife 2:e00471; Segal (2013) eLife 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments, the Cas nuclease has DNA cleavage activity. The Cas nuclease can direct cleavage of one or both strands at a location in a target DNA sequence. For example, the Cas nuclease can be a nickase having one or more inactivated catalytic domains that cleaves a single strand of a target DNA sequence.

Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP 269215, and the amino acid sequence of Streptococcus thermophilus wild-type Cas9 polypeptide is set forth, e.g., in NBCI Ref. Seq. No. WP_011681470. CRISPR-related endonucleases that are useful in the present invention are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia mucimphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cas9” refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g., the two catalytic domains are derived from different bacteria species.

Useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of INDEL mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme can contain a mutation at D10, E762, H983 or D986, as well as a mutation at H840 or N863. In some instances, the dCas9 enzyme contains a D10A or D10N mutation. Also, the dCas9 enzyme can include a H840A, H840Y, or H840N. In some embodiments, the dCas9 enzyme of the present invention comprises D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target DNA.

For genome editing methods, the Cas nuclease can be a Cas9 fusion protein such as a polypeptide comprising the catalytic domain of the type IIS restriction enzyme, FokI, linked to dCas9. The FokI-dCas9 fusion protein (fCas9) can use two guide RNAs to bind to a single strand of target DNA to generate a double-strand break.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) [also referred to as eSpCas9(1.0)], and SpCas9 (K848A/K1003A/R1060A) [also referred to as eSpCas9(1.1)] variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

As described in the examples, a CRISPR/Cas nuclease system was used to gene edit sinus basal cells expressing Krt5 that were isolated from patients having CF. The cells were electroporated with Cas9 ribonuclear protein (RNP) and MS-sgRNA (sgRNA modified with 2′-O-methyl 3′phosphorothioate (MS) in the 5′ and 3′ terminal nucleotides), followed by incubation with AAV6 containing a codon diverged sequence from CFTR exon 11 that includes the ΔF508 region in the CFTR protein.

A homologous recombination in 43±5% alleles was achieved in non-CF sinus basal cells. Further, 34±4% and 41±15% corrections, respectively, were achieved in sinus basal cells from homozygous and compound heterozygous patients. On differentiation in air-liquid interfaces (ALI), up to 75% (range=4-75%) restoration of CFTRᵢₙₕ-172 sensitive Cl⁻ current relative to non-CF controls was observed. This level of CFTR function compares favorably with the 2-20% CFTR function observed in patients with milder class IV mutations associated with lower mortality (e.g., R117H-7T in the CFTR protein).

Zinc Finger Nucleases (ZFNs)

“Zinc finger nucleases” or “ZFNs” are a fusion between the cleavage domain of FokI and a DNA recognition domain containing 3 or more zinc finger motifs. The heterodimerization at a particular position in the DNA of two individual ZFNs in precise orientation and spacing leads to a double-strand break in the DNA. In some cases, ZFNs fuse a cleavage domain to the C-terminus of each zinc finger domain. In order to allow the two cleavage domains to dimerize and cleave DNA, the two individual ZFNs bind opposite strands of DNA with their C-termini at a certain distance apart. In some cases, linker sequences between the zinc finger domain and the cleavage domain requires the 5′ edge of each binding site to be separated by about 5-7 bp. Exemplary ZFNs that are useful in the present invention include, but are not limited to, those described in Umov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

ZFNs can generate a double-strand break in a target DNA, resulting in DNA break repair which allows for the introduction of gene modification. DNA break repair can occur via non-homologous end joining (NHEJ) or homology-directed repair (HDR). In HDR, a donor DNA repair template that contains homology arms flanking sites of the target DNA can be provided.

In some embodiments, a ZFN is a zinc finger nickase which can be an engineered ZFN that induces site-specific single-strand DNA breaks or nicks, thus resulting in HDR. Descriptions of zinc finger nickases are found, e.g., in Ramirez et al., Nucl Acids Res, 2012, 40(12):5560-8; Kim et al., Genome Res, 2012, 22(7):1327-33.

TALENs

“TALENs” or “TAL-effector nucleases” are engineered transcription activator-like effector nucleases that contain a central domain of DNA-binding tandem repeats, a nuclear localization signal, and a C-terminal transcriptional activation domain. In some instances, a DNA-binding tandem repeat comprises 33-35 amino acids in length and contains two hypervariable amino acid residues at positions 12 and 13 that can recognize one or more specific DNA base pairs. TALENs can be produced by fusing a TAL effector DNA binding domain to a DNA cleavage domain. For instance, a TALE protein may be fused to a nuclease such as a wild-type or mutated FokI endonuclease or the catalytic domain of FokI. Several mutations to FokI have been made for its use in TALENs, which, for example, improve cleavage specificity or activity. Such TALENs can be engineered to bind any desired DNA sequence.

TALENs can be used to generate gene modifications by creating a double-strand break in a target DNA sequence, which in turn, undergoes NHEJ or HDR. In some cases, a single-stranded donor DNA repair template is provided to promote HDR.

Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55.

Meganucleases

“Meganucleases” are rare-cutting endonucleases or homing endonucleases that can be highly specific, recognizing DNA target sites ranging from at least 12 base pairs in length, e.g., from 12 to 40 base pairs or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric.

In some instances, the meganuclease is naturally-occurring (found in nature) or wild-type, and in other instances, the meganuclease is non-natural, artificial, engineered, synthetic, rationally designed, or man-made. In certain embodiments, the meganuclease of the present invention includes an I-CreI meganuclease, I-CeuI meganuclease, I-MsoI meganuclease, I-SceI meganuclease, variants thereof, mutants thereof, and derivatives thereof.

Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Natl Acad Sci USA, 2014, 111 (11): 4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

### VII. DNA-Targeting RNA

In some embodiments, the methods of the present disclosure comprise introducing into an airway stem cell a guide nucleic acid, e.g., DNA-targeting RNA (e.g., a single guide RNA (sgRNA) or a double guide nucleic acid) or a nucleotide sequence encoding the guide nucleic acid (e.g., DNA-targeting RNA). In particular embodiments, a single guide RNA (sgRNA) comprising a first nucleotide sequence that is complementary to a target nucleic acid and a second nucleotide sequence that interacts with a CRISPR-associated protein (Cas) polypeptide is introduced into an airway stem cell. In some embodiments, an sgRNA includes at least one modified nucleotide.

The DNA-targeting RNA (e.g., sgRNA) can comprise a first nucleotide sequence that is complementary to a specific sequence within a target DNA (e.g., a guide sequence) and a second nucleotide sequence comprising a protein-binding sequence that interacts with a DNA nuclease (e.g., Cas9 nuclease) or a variant thereof (e.g., a scaffold sequence or tracrRNA). The guide sequence (“first nucleotide sequence”) of a DNA-targeting RNA can comprise about 10 to about 2000 nucleic acids, for example, about 10 to about 100 nucleic acids, about 10 to about 500 nucleic acids, about 10 to about 1000 nucleic acids, about 10 to about 1500 nucleic acids, about 10 to about 2000 nucleic acids, about 50 to about 100 nucleic acids, about 50 to about 500 nucleic acids, about 50 to about 1000 nucleic acids, about 50 to about 1500 nucleic acids, about 50 to about 2000 nucleic acids, about 100 to about 500 nucleic acids, about 100 to about 1000 nucleic acids, about 100 to about 1500 nucleic acids, about 100 to about 2000 nucleic acids, about 500 to about 1000 nucleic acids, about 500 to about 1500 nucleic acids, about 500 to about 2000 nucleic acids, about 1000 to about 1500 nucleic acids, about 1000 to about 2000 nucleic acids, or about 1500 to about 2000 nucleic acids at the 5′ end that can direct the DNA nuclease (e.g., Cas9 nuclease) to the target DNA site using RNA-DNA complementarity base pairing. In some embodiments, the guide sequence of a DNA-targeting RNA comprises about 100 nucleic acids at the 5′ end that can direct the DNA nuclease (e.g., Cas9 nuclease) to the target DNA site using RNA-DNA complementarity base pairing. In some embodiments, the guide sequence comprises 20 nucleic acids at the 5′ end that can direct the DNA nuclease (e.g., Cas9 nuclease) to the target DNA site using RNA-DNA complementarity base pairing. In other embodiments, the guide sequence comprises less than 20, e.g., 19, 18, 17, 16, 15 or less, nucleic acids that are complementary to the target DNA site. The guide sequence can include 17 nucleic acids that can direct the DNA nuclease (e.g., Cas9 nuclease) to the target DNA site. In some instances, the guide sequence contains about 1 to about 10 nucleic acid mismatches in the complementarity region at the 5′ end of the targeting region. In other instances, the guide sequence contains no mismatches in the complementarity region at the last about 5 to about 12 nucleic acids at the 3′ end of the targeting region.

The protein-binding scaffold sequence (“second nucleotide sequence”) of the DNA-targeting RNA (e.g., sgRNA) can comprise two complementary stretches of nucleotides that hybridize to one another to form a double-stranded RNA duplex (dsRNA duplex). The protein-binding scaffold sequence can be between about 30 nucleic acids to about 200 nucleic acids, e.g., about 40 nucleic acids to about 200 nucleic acids, about 50 nucleic acids to about 200 nucleic acids, about 60 nucleic acids to about 200 nucleic acids, about 70 nucleic acids to about 200 nucleic acids, about 80 nucleic acids to about 200 nucleic acids, about 90 nucleic acids to about 200 nucleic acids, about 100 nucleic acids to about 200 nucleic acids, about 110 nucleic acids to about 200 nucleic acids, about 120 nucleic acids to about 200 nucleic acids, about 130 nucleic acids to about 200 nucleic acids, about 140 nucleic acids to about 200 nucleic acids, about 150 nucleic acids to about 200 nucleic acids, about 160 nucleic acids to about 200 nucleic acids, about 170 nucleic acids to about 200 nucleic acids, about 180 nucleic acids to about 200 nucleic acids, or about 190 nucleic acids to about 200 nucleic acids. In certain aspects, the protein-binding sequence can be between about 30 nucleic acids to about 190 nucleic acids, e.g., about 30 nucleic acids to about 180 nucleic acids, about 30 nucleic acids to about 170 nucleic acids, about 30 nucleic acids to about 160 nucleic acids, about 30 nucleic acids to about 150 nucleic acids, about 30 nucleic acids to about 140 nucleic acids, about 30 nucleic acids to about 130 nucleic acids, about 30 nucleic acids to about 120 nucleic acids, about 30 nucleic acids to about 110 nucleic acids, about 30 nucleic acids to about 100 nucleic acids, about 30 nucleic acids to about 90 nucleic acids, about 30 nucleic acids to about 80 nucleic acids, about 30 nucleic acids to about 70 nucleic acids, about 30 nucleic acids to about 60 nucleic acids, about 30 nucleic acids to about 50 nucleic acids, or about 30 nucleic acids to about 40 nucleic acids.

In some embodiments, the DNA-targeting RNA (e.g., sgRNA) is a truncated form thereof comprising a guide sequence having a shorter region of complementarity to a target DNA sequence (e.g., less than 20 nucleotides in length). In certain instances, the truncated DNA-targeting RNA (e.g., sgRNA) provides improved DNA nuclease (e.g., Cas9 nuclease) specificity by reducing off-target effects. For example, a truncated sgRNA can comprise a guide sequence having 17, 18, or 19 complementary nucleotides to a target DNA sequence (e.g., 17-18, 17-19, or 18-19 complementary nucleotides). See, e.g., Fu et al., Nat. Biotechnol., 32(3): 279-284 (2014).

The DNA-targeting RNA (e.g., sgRNA) can be selected using any of the web-based software described above. As a non-limiting example, considerations for selecting a DNA-targeting RNA can include the PAM sequence for the Cas9 nuclease to be used, and strategies for minimizing off-target modifications. Tools, such as the CRISPR Design Tool, can provide sequences for preparing the DNA-targeting RNA, for assessing target modification efficiency, and/or assessing cleavage at off-target sites.

The DNA-targeting RNA (e.g., sgRNA) can be produced by any method known to one of ordinary skill in the art. In some embodiments, a nucleotide sequence encoding the DNA-targeting RNA is cloned into an expression cassette or an expression vector. In certain embodiments, the nucleotide sequence is produced by PCR and contained in an expression cassette. For instance, the nucleotide sequence encoding the DNA-targeting RNA can be PCR amplified and appended to a promoter sequence, e.g., a U6 RNA polymerase III promoter sequence. In other embodiments, the nucleotide sequence encoding the DNA-targeting RNA is cloned into an expression vector that contains a promoter, e.g., a U6 RNA polymerase III promoter, and a transcriptional control element, enhancer, U6 termination sequence, one or more nuclear localization signals, etc. In some embodiments, the expression vector is multicistronic or bicistronic and can also include a nucleotide sequence encoding a fluorescent protein, an epitope tag and/or an antibiotic resistance marker. In certain instances of the bicistronic expression vector, the first nucleotide sequence encoding, for example, a fluorescent protein, is linked to a second nucleotide sequence encoding, for example, an antibiotic resistance marker using the sequence encoding a self-cleaving peptide, such as a viral 2A peptide. Viral 2A peptides including foot-and-mouth disease virus 2A (F2A); equine rhinitis A virus 2A (E2A); porcine teschovirus-1 2A (P2A) and Thoseaasigna virus 2A (T2A) have high cleavage efficiency such that two proteins can be expressed simultaneously yet separately from the same RNA transcript.

Suitable expression vectors for expressing the DNA-targeting RNA (e.g., sgRNA) are commercially available from Addgene, Sigma-Aldrich, and Life Technologies. The expression vector can be pLQ1651 (Addgene Catalog No. 51024) which includes the fluorescent protein mCherry. Non-limiting examples of other expression vectors include pX330, pSpCas9, pSpCas9n, pSpCas9-2A-Puro, pSpCas9-2A-GFP, pSpCas9n-2A-Puro, the GeneArt® CRISPR Nuclease OFP vector, the GeneArt® CRISPR Nuclease OFP vector, and the like.

In certain embodiments, the DNA-targeting RNA (e.g., sgRNA) is chemically synthesized. DNA-targeting RNAs can be synthesized using 2′-O-thionocarbamate-protected nucleoside phosphoramidites. Methods are described in, e.g., Dellinger et al., J. American Chemical Society 133, 11540-11556 (2011); Threlfall et al., Organic & Biomolecular Chemistry 10, 746-754 (2012); and Dellinger et al., J. American Chemical Society 125, 940-950 (2003).

In particular embodiments, the DNA-targeting RNA (e.g., sgRNA) is chemically modified. As a non-limiting example, the DNA-targeting RNA is a modified sgRNA comprising a first nucleotide sequence complementary to a target nucleic acid (e.g., a guide sequence or crRNA) and a second nucleotide sequence that interacts with a Cas polypeptide (e.g., a scaffold sequence or tracrRNA).

Without being bound by any particular theory, sgRNAs containing one or more chemical modifications can increase the activity, stability, and specificity and/or decrease the toxicity of the modified sgRNA compared to a corresponding unmodified sgRNA when used for CRISPR-based genome editing, e.g., homologous recombination. Non-limiting advantages of modified sgRNAs include greater ease of delivery into target cells, increased stability, increased duration of activity, and reduced toxicity. The modified sgRNAs can provide higher frequencies of on-target genome editing (e.g., homologous recombination), improved activity, and/or specificity compared to their unmodified sequence equivalents.

One or more nucleotides of the guide sequence and/or one or more nucleotides of the scaffold sequence can be a modified nucleotide. For instance, a guide sequence that is about 20 nucleotides in length may have 1 or more, e.g., 1, 2, 3, 4, 5, 6, 7, 8, 9, 10, 11, 12, 13, 14, 15, 16, 17, 18, 19, or 20 modified nucleotides. In some cases, the guide sequence includes at least 2, 3, 4, 5, 6, 7, 8, 9, 10, or more modified nucleotides. In other cases, the guide sequence includes at least 2, 3, 4, 5, 6, 7, 8, 9, 10, 11, 12, 13, 14, 15, 16, 17, 19, 20, or more modified nucleotides. The modified nucleotide can be located at any nucleic acid position of the guide sequence. In other words, the modified nucleotides can be at or near the first and/or last nucleotide of the guide sequence, and/or at any position in between. For example, for a guide sequence that is 20 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, and/or position 20 of the guide sequence. In certain instances, from about 10% to about 30%, e.g., about 10% to about 25%, about 10% to about 20%, about 10% to about 15%, about 15% to about 30%, about 20% to about 30%, or about 25% to about 30% of the guide sequence can comprise modified nucleotides. In other instances, from about 10% to about 30%, e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% of the guide sequence can comprise modified nucleotides.

In certain embodiments, the modified nucleotides are located at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and/or at internal positions within the guide sequence.

In some embodiments, the scaffold sequence of the modified sgRNA contains one or more modified nucleotides. For example, a scaffold sequence that is about 80 nucleotides in length may have 1 or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 76, 77, 78, 79, or 80 modified nucleotides. In some instances, the scaffold sequence includes at least 2, 3, 4, 5, 6, 7, 8, 9, 10, or more modified nucleotides. In other instances, the scaffold sequence includes at least 2, 3, 4, 5, 6, 7, 8, 9, 10, 11, 12, 13, 14, 15, 16, 17, 19, 20, or more modified nucleotides. The modified nucleotides can be located at any nucleic acid position of the scaffold sequence. For example, the modified nucleotides can be at or near the first and/or last nucleotide of the scaffold sequence, and/or at any position in between. For example, for a scaffold sequence that is about 80 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, position 37, position 38, position 39, position 40, position 41, position 42, position 43, position 44, position 45, position 46, position 47, position 48, position 49, position 50, position 51, position 52, position 53, position 54, position 55, position 56, position 57, position 58, position 59, position 60, position 61, position 62, position 63, position 64, position 65, position 66, position 67, position 68, position 69, position 70, position 71, position 72, position 73, position 74, position 75, position 76, position 77, position 78, position 79, and/or position 80 of the sequence. In some instances, from about 1% to about 10%, e.g., about 1% to about 8%, about 1% to about 5%, about 5% to about 10%, or about 3% to about 7% of the scaffold sequence can comprise modified nucleotides. In other instances, from about 1% to about 10%, e.g., about 1%, about 2%, about 3%, about 4%, about 5%, about 6%, about 7%, about 8%, about 9%, or about 10% of the scaffold sequence can comprise modified nucleotides.

In certain embodiments, the modified nucleotides are located at the 3′-end (e.g., the terminal nucleotide at the 3′-end) or near the 3′-end (e.g., within 1, 2, 3, 4, or 5 nucleotides of the 3′-end) of the scaffold sequence and/or at internal positions within the scaffold sequence.

In some embodiments, the modified sgRNA comprises one, two, or three consecutive or non-consecutive modified nucleotides starting at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and one, two, or three consecutive or non-consecutive modified nucleotides starting at the 3′-end (e.g., the terminal nucleotide at the 3′-end) or near the 3′-end (e.g., within 1, 2, 3, 4, or 5 nucleotides of the 3′-end) of the scaffold sequence.

In some instances, the modified sgRNA comprises one modified nucleotide at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and one modified nucleotide at the 3′-end (e.g., the terminal nucleotide at the 3′-end) or near the 3′-end (e.g., within 1, 2, 3, 4, or 5 nucleotides of the 3′-end) of the scaffold sequence.

In other instances, the modified sgRNA comprises two consecutive or non-consecutive modified nucleotides starting at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and two consecutive or non-consecutive modified nucleotides starting at the 3′-end (e.g., the terminal nucleotide at the 3′-end) or near the 3′-end (e.g., within 1, 2, 3, 4, or 5 nucleotides of the 3′-end) of the scaffold sequence.

In yet other instances, the modified sgRNA comprises three consecutive or non-consecutive modified nucleotides starting at the 5′-end (e.g., the terminal nucleotide at the 5′-end) or near the 5′-end (e.g., within 1, 2, 3, 4, or 5 nucleotides of the terminal nucleotide at the 5′-end) of the guide sequence and three consecutive or non-consecutive modified nucleotides starting at the 3′-end (e.g., the terminal nucleotide at the 3′-end) or near the 3′-end (e.g., within 1, 2, 3, 4, or 5 nucleotides of the 3′-end) of the scaffold sequence.

In particular embodiments, the modified sgRNA comprises three consecutive modified nucleotides at the 5′-end of the guide sequence and three consecutive modified nucleotides at the 3′-end of the scaffold sequence.

The modified nucleotides of the sgRNA can include a modification in the ribose (e.g., sugar) group, phosphate group, nucleobase, or any combination thereof. In some embodiments, the modification in the ribose group comprises a modification at the 2′ position of the ribose.

In some embodiments, the modified nucleotide includes a 2′fluoro-arabino nucleic acid, tricycle-DNA (tc-DNA), peptide nucleic acid, cyclohexene nucleic acid (CeNA), locked nucleic acid (LNA), ethylene-bridged nucleic acid (ENA), a phosphodiamidate morpholino, or a combination thereof.

Modified nucleotides or nucleotide analogues can include sugar- and/or backbone-modified ribonucleotides (i.e., include modifications to the phosphate-sugar backbone). For example, the phosphodiester linkages of a native or natural RNA may be modified to include at least one of a nitrogen or sulfur heteroatom. In some backbone-modified ribonucleotides, the phosphoester group connecting to adjacent ribonucleotides may be replaced by a modified group, e.g., a phosphothioate group. In preferred sugar-modified ribonucleotides, the 2′ moiety is a group selected from H, OR, R, halo, SH, SR, NH₂, NHR, NR₂ or ON, wherein R is C₁-C₆ alkyl, alkenyl or alkynyl and halo is F, Cl, Br or I.

In some embodiments, the modified nucleotide contains a sugar modification. Non-limiting examples of sugar modifications include 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-O-alkyl oligoribonucleotide, 2′-deoxy-2′-C-alkyl oligoribonucleotide (2′-O-methylcytidine-5′-triphosphate, 2′-methyluridine-5′-triphosphate), 2′-C-alkyl oligoribonucleotide, and isomers thereof (2′-aracytidine-5′-triphosphate, 2′-arauridine-5′-triphosphate), azidotriphosphate (2′-azido-2′-deoxycytidine-5′-triphosphate, 2′-azido-2′-deoxyuridine-5′-triphosphate), and combinations thereof.

In some embodiments, the modified sgRNA contains one or more 2′-fluoro, 2′-amino and/or 2′-thio modifications. In some instances, the modification is a 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, 5-amino-allyl-uridine, 5-bromo-uridine, 5-iodo-uridine, 5-methyl-cytidine, ribo-thymidine, 2-aminopurine, 2′-amino-butyryl-pyrene-uridine, 5-fluoro-cytidine, and/or 5-fluoro-uridine.

There are more than 96 naturally occurring nucleoside modifications found on mammalian RNA. See, e.g., Limbach et al., Nucleic Acids Research, 22(12):2183-2196 (1994). The preparation of nucleotides and modified nucleotides and nucleosides are well-known in the art, e.g., from U.S. Pat. Nos. 4,373,071, 4,458,066, 4,500,707, 4,668,777, 4,973,679, 5,047,524, 5,132,418, 5,153,319, 5,262,530, and 5,700,642. Numerous modified nucleosides and modified nucleotides that are suitable for use are commercially available. The nucleoside can be an analogue of a naturally occurring nucleoside. In some cases, the analogue is dihydrouridine, methyladenosine, methylcytidine, methyluridine, methylpseudouridine, thiouridine, deoxycytodine, and deoxyuridine.

In some cases, the modified sgRNA includes a nucleobase-modified ribonucleotide, i.e., a ribonucleotide containing at least one non-naturally occurring nucleobase instead of a naturally occurring nucleobase. Non-limiting examples of modified nucleobases which can be incorporated into modified nucleosides and modified nucleotides include m5C (5-methylcytidine), m5U (5-methyluridine), m6A (N6-methyladenosine), s2U (2-thiouridine), Um (2′-O-methyluridine), m1A (1-methyl adenosine), m2A (2-methyladenosine), Am (2-1-O-methyladenosine), ms2m6A (2-methylthio-N6-methyladenosine), i6A (N6-isopentenyl adenosine), ms2i6A (2-methylthio-N6isopentenyladenosine), io6A (N6-(cis-hydroxyisopentenyl) adenosine), ms2io6A (2-methylthio-N6-(cis-hydroxyisopentenyl)adenosine), g6A (N6-glycinylcarbamoyladenosine), t6A (N6-threonyl carbamoyladenosine), ms2t6A (2-methylthio-N6-threonyl carbamoyladenosine), m6t6A (N6-methyl-N6-threonylcarbamoyladenosine), hn6A (N6.-hydroxynorvalylcarbamoyl adenosine), ms2hn6A (2-methylthio-N6-hydroxynorvalyl carbamoyladenosine), Ar(p) (2′-O-ribosyladenosine(phosphate)), I (inosine), m11 (1-methylinosine), m'Im (1,2′-O-dimethylinosine), m3C (3-methylcytidine), Cm (2T-O-methylcytidine), s2C (2-thiocytidine), ac4C (N4-acetylcytidine), f5C (5-fonnylcytidine), m5Cm (5,2-O-dimethylcytidine), ac4Cm (N4acetyl2TOmethylcytidine), k2C (lysidine), m1G (1-methylguanosine), m2G (N2-methylguanosine), m7G (7-methylguanosine), Gm (2′-O-methylguanosine), m22G (N2,N2-dimethylguanosine), m2Gm (N2,2′-O-dimethylguanosine), m22Gm (N2,N2,2′-O-trimethylguanosine), Gr(p) (2′-O-ribosylguanosine(phosphate)), yW (wybutosine), o2yW (peroxywybutosine), OHyW (hydroxywybutosine), OHyW* (undermodified hydroxywybutosine), imG (wyosine), mimG (methylguanosine), Q (queuosine), oQ (epoxyqueuosine), galQ (galtactosyl-queuosine), manQ (mannosyl-queuosine), preQo (7-cyano-7-deazaguanosine), preQi (7-aminomethyl-7-deazaguanosine), G (archaeosine), D (dihydrouridine), m5Um (5,2′-O-dimethyluridine), s4U (4-thiouridine), m5s2U (5-methyl-2-thiouridine), s2Um (2-thio-2′-O-methyluridine), acp3U (3-(3-amino-3-carboxypropyl)uridine), ho5U (5-hydroxyuridine), mo5U (5-methoxyuridine), cmo5U (uridine 5-oxyacetic acid), mcmo5U (uridine 5-oxyacetic acid methyl ester), chm5U (5-(carboxyhydroxymethyl)uridine)), mchm5U (5-(carboxyhydroxymethyl)uridine methyl ester), mcm5U (5-methoxycarbonyl methyluridine), mcm5Um (S-methoxycarbonylmethyl-2-O-methyluridine), mcm5s2U (5-methoxycarbonylmethyl-2-thiouridine), nm5s2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyl uridine), ncm5Um (5-carbamoylmethyl-2′-O-methyluridine), cmnm5U (5-carboxymethylaminomethyluridine), cnmm5Um (5-carboxymethylaminomethyl-2-L-Omethyluridine), cmnm5s2U (5-carboxymethylaminomethyl-2-thiouridine), m62A (N6,N6-dimethyladenosine), Tm (2′-O-methylinosine), m4C (N4-methylcytidine), m4Cm (N4,2-O-dimethylcytidine), hm5C (5-hydroxymethylcytidine), m3U (3-methyluridine), cm5U (5-carboxymethyluridine), m6Am (N6,T-O-dimethyladenosine), rn62Am (N6,N6,O-2-trimethyladenosine), m2′7G (N2,7-dimethylguanosine), m2′2′7G (N2,N2,7-trimethylguanosine), m3Um (3,2T-O-dimethyluridine), m5D (5-methyldihydrouridine), f5Cm (5-formyl-2′-O-methylcytidine), m1Gm (1,2′-O-dimethylguanosine), m′Am (1,2-O-dimethyl adenosine)irinomethyluridine), tm5s2U (S-taurinomethyl-2-thiouridine)), imG-14 (4-demethyl guanosine), imG2 (isoguanosine), or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyOuracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C₂-C₆)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, and combinations thereof.

In some embodiments, the phosphate backbone of the modified sgRNA is altered. The modified sgRNA can include one or more phosphorothioate, phosphoramidate (e.g., N3′-P5′-phosphoramidate (NP)), 2′-O-methoxy-ethyl (2′MOE), 2′-O-methyl-ethyl (2′ME), and/or methylphosphonate linkages. In certain instances, the phosphate group is changed to a phosphothioate, 2′-O-methoxy-ethyl (2′MOE), 2′-O-methyl-ethyl (2′ME), N3′-P5′-phosphoramidate (NP), and the like.

In particular embodiments, the modified nucleotide comprises a 2′-O-methyl nucleotide (M), a 2′-O-methyl, 3′-phosphorothioate nucleotide (MS), a 2′-O-methyl, 3′thioPACE nucleotide (MSP), or a combination thereof.

In some instances, the modified sgRNA includes one or more MS nucleotides. In other instances, the modified sgRNA includes one or more MSP nucleotides. In yet other instances, the modified sgRNA includes one or more MS nucleotides and one or more MSP nucleotides. In further instances, the modified sgRNA does not include M nucleotides. In certain instances, the modified sgRNA includes one or more MS nucleotides and/or one or more MSP nucleotides, and further includes one or more M nucleotides. In certain other instances, MS nucleotides and/or MSP nucleotides are the only modified nucleotides present in the modified sgRNA.

It should be noted that any of the modifications described herein may be combined and incorporated in the guide sequence and/or the scaffold sequence of the modified sgRNA.

In some cases, the modified sgRNAs also include a structural modification such as a stem loop, e.g., M2 stem loop or tetraloop.

The chemically modified sgRNAs can be used with any CRISPR-associated or RNA-guided technology. As described herein, the modified sgRNAs can serve as a guide for any Cas9 polypeptide or variant thereof, including any engineered or man-made Cas9 polypeptide. The modified sgRNAs can target DNA and/or RNA molecules in isolated cells or in vivo (e.g., in an animal).

### VIII. Recombinant Donor Adeno-Associated Viral (AAV) Vectors

Provided herein is a homologous donor adeno-associated viral (AAV) vector comprising a recombinant donor template comprising two nucleotide sequences comprising two non-overlapping, homologous portions of the target nucleic acid (“homology arms”), wherein the nucleotide sequences are located at the 5′ and 3′ ends of a nucleotide sequence corresponding to the target nucleic acid to undergo homologous recombination. In some embodiments, the donor template can further comprise a selectable marker, a detectable marker, and/or a cell purification marker.

In some embodiments, the homology arms are the same length. In other embodiments, the homology arms are different lengths. The homology arms can be at least about 10 base pairs (bp), e.g., at least about 10 bp, 15 bp, 20 bp, 25 bp, 30 bp, 35 bp, 45 bp, 55 bp, 65 bp, 75 bp, 85 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or longer. The homology arms can be about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb.

The recombinant donor template can be introduced or delivered into an airway stem cell via viral gene transfer. In some embodiments, the donor template is delivered using an adeno-associated virus (AAV). Any AAV serotype, e.g., human AAV serotype, can be used including, but not limited to, AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 11 (AAV11), a variant thereof, or a shuffled variant thereof (e.g., a chimeric variant thereof). In some embodiments, an AAV variant has at least 90%, e.g., 90%, 91%, 92%, 93%, 94%, 95%, 96%, 97%, 98%, 99% or more amino acid sequence identity to a wild-type AAV. An AAV1 variant can have at least 90%, e.g., 90%, 91%, 92%, 93%, 94%, 95%, 96%, 97%, 98%, 99% or more amino acid sequence identity to a wild-type AAV1. An AAV2 variant can have at least 90%, e.g., 90%, 91%, 92%, 93%, 94%, 95%, 96%, 97%, 98%, 99% or more amino acid sequence identity to a wild-type AAV2. An AAV3 variant can have at least 90%, e.g., 90%, 91%, 92%, 93%, 94%, 95%, 96%, 97%, 98%, 99% or more amino acid sequence identity to a wild-type AAV3. An AAV4 variant can have at least 90%, e.g., 90%, 91%, 92%, 93%, 94%, 95%, 96%, 97%, 98%, 99% or more amino acid sequence identity to a wild-type AAV4. An AAV5 variant can have at least 90%, e.g., 90%, 91%, 92%, 93%, 94%, 95%, 96%, 97%, 98%, 99% or more amino acid sequence identity to a wild-type AAV5. An AAV6 variant can have at least 90%, e.g., 90%, 91%, 92%, 93%, 94%, 95%, 96%, 97%, 98%, 99% or more amino acid sequence identity to a wild-type AAV6. An AAV7 variant can have at least 90%, e.g., 90%, 91%, 92%, 93%, 94%, 95%, 96%, 97%, 98%, 99% or more amino acid sequence identity to a wild-type AAV7. An AAV8 variant can have at least 90%, e.g., 90%, 91%, 92%, 93%, 94%, 95%, 96%, 97%, 98%, 99% or more amino acid sequence identity to a wild-type AAV8. An AAV9 variant can have at least 90%, e.g., 90%, 91%, 92%, 93%, 94%, 95%, 96%, 97%, 98%, 99% or more amino acid sequence identity to a wild-type AAV9. An AAV10 variant can have at least 90%, e.g., 90%, 91%, 92%, 93%, 94%, 95%, 96%, 97%, 98%, 99% or more amino acid sequence identity to a wild-type AAV10. An AAV11 variant can have at least 90%, e.g., 90%, 91%, 92%, 93%, 94%, 95%, 96%, 97%, 98%, 99% or more amino acid sequence identity to a wild-type AAV11. An AAV12 variant can have at least 90%, e.g., 90%, 91%, 92%, 93%, 94%, 95%, 96%, 97%, 98%, 99% or more amino acid sequence identity to a wild-type AAV12.

In some instances, one or more regions of at least two different AAV serotype viruses are shuffled and reassembled to generate an AAV chimera virus. For example, a chimeric AAV can comprise inverted terminal repeats (ITRs) that are of a heterologous serotype compared to the serotype of the capsid. The resulting chimeric AAV virus can have a different antigenic reactivity or recognition, compared to its parental serotypes. In some embodiments, a chimeric variant of an AAV includes amino acid sequences from 2, 3, 4, 5, or more different AAV serotypes.

Descriptions of AAV variants and methods for generating thereof are found, e.g., in Weitzman and Linden. Chapter 1-Adeno-Associated Virus Biology in Adeno-Associated Virus: Methods and Protocols Methods in Molecular Biology, vol. 807. Snyder and Moullier, eds., Springer, 2011; Potter et al., Molecular Therapy—Methods & Clinical Development, 2014, 1, 14034; Bartel et al., Gene Therapy, 2012, 19, 694-700; Ward and Walsh, Virology, 2009, 386(2):237-248; and Li et al., Mol Ther, 2008, 16(7):1252-1260. AAV virions (e.g., viral vectors or viral particle) can be transduced into airway stem cells to introduce the recombinant donor template into the cell. A recombinant donor template can be packaged into an AAV viral vector according to any method known to those skilled in the art. Examples of useful methods are described in McClure et al., J Vis Exp, 2001, 57:3378.

The recombinant donor template may comprise two nucleotide sequences that include two non-overlapping, homologous region of the target nucleic acid. The nucleotide sequences are sequences that are homologous to the genomic sequences flanking the site-specific double-strand break (DSB) generated by the engineered nuclease system of the present invention, e.g., an sgRNA and a Cas polypeptide. The two nucleotide sequences are located at the 5′ and 3′ ends of a nucleotide sequence that corresponds to the target nucleic acid. The donor template is used by the engineered nuclease to repair the DSB and provide precise nucleotide changes at the site of the break.

The recombinant donor template of interest can also include one or more nucleotide sequences encoding a functional polypeptide or a fragment thereof. The donor template can be used to introduce a precise and specific nucleotide substitution or deletion in a pre-selected gene, or in some cases, a transgene. Any of a number of transcription and translation control elements, including promoter, transcription enhancers, transcription terminators, and the like, may be used in the donor template. In some embodiments, the recombinant donor template of interest includes a promoter. In other embodiments, the recombinant donor template of interest is promoterless. Useful promoters can be derived from viruses, or any organism, e.g., prokaryotic or eukaryotic organisms. Suitable promoters include, but are not limited to, the spleen focus-forming virus promoter (SFFV), elongation factor-1 alpha promoter (EF1α), Ubiquitin C promoter (UbC), phosphoglycerate kinase promoter (PGK), simian virus 40 (SV40) early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc.

In some embodiments, the recombinant donor template further comprises one or more sequences encoding polyadenylation (polyA) signals. Suitable polyA signals include, but are not limited to, SV40 polyA, thymidine kinase (TK) polyA, bovine growth hormone (BGH) polyA, human growth hormone (hGH) polyA, rabbit beta globin (rbGlob) polyA, or a combination thereof. The donor template can also further comprise a non-polyA transcript-stabilizing element (e.g., woodchuck hepatitis virus posttranscriptional regulatory element (WPRE)) or a nuclear export element (e.g., constitutive transport element (CTE)).

In some embodiments, the transgene is a detectable marker or a cell surface marker. In certain instances, the detectable marker is a fluorescent protein such as green fluorescent protein (GFP), enhanced green fluorescent protein (EGFP), red fluorescent protein (RFP), blue fluorescent protein (BFP), cyan fluorescent protein (CFP), yellow fluorescent protein (YFP), mCherry, tdTomato, DsRed-Monomer, DsRed-Express, DSRed-Express2, DsRed2, AsRed2, mStrawberry, mPlum, mRaspberry, HcRedl, E2-Crimson, mOrange, mOrange2, mBanana, ZsYellowl, TagBFP, mTagBFP2, Azurite, EBFP2, mKalamal, Sirius, Sapphire, T-Sapphire, ECFP, Cerulean, SCFP3A, mTurquoise, mTurquoise2, monomeric Midoriishi-Cyan, TagCFP, mTFP1, Emerald, Superfolder GFP, Monomeric Azami Green, TagGFP2, mUKG, mWasabi, Clover, mNeonGreen, Citrine, Venus, SYFP2, TagYFP, Monomeric Kusabira-Orange, mKOk, mKO2, mTangerine, mApple, mRuby, mRuby2, HcRed-Tandem, mKate2, mNeptune, NiFP, mKeima Red, LSS-mKate1, LSS-mKate2, mBeRFP, PA-GFP, PAmCherryl, PATagRFP, TagRFP6457, IFP1.2, iRFP, Kaede (green), Kaede (red), KikGR1 (green), KikGR1 (red), PS-CFP2, mEos2 (green), mEos2 (red), mEos3.2 (green), mEos3.2 (red), PSmOrange, Dronpa, Dendra2, Timer, AmCyanl, or a combination thereof. In other instances, the cell surface marker is a marker not normally expressed on the cells such as a truncated nerve growth factor receptor (tNGFR), a truncated epidermal growth factor receptor (tEGFR), CD8, truncated CD8, CD19, truncated CD19, a variant thereof, a fragment thereof, a derivative thereof, or a combination thereof.

### IX. Introducing DNA Nucleases, Modified sgRNAs, and Homologous Donor AAV Vectors into Airway Stem Cells

Methods for introducing polypeptides, nucleic acids, and viral vectors (e.g., viral particles) into a target cell (e.g., an airway stem cell) are known in the art. Any known method can be used to introduce a polypeptide or a nucleic acid (e.g., a nucleotide sequence encoding the DNA nuclease or a modified sgRNA) into a target cell (e.g., an airway stem cell). Non-limiting examples of suitable methods include electroporation (e.g., nucleofection), viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

Any known method can be used to introduce a viral vector (e.g., viral particle) into a target cell (e.g., an airway stem cell). In some embodiments, the homologous donor adeno-associated viral (AAV) vector described herein is introduced into a target cell (e.g., an airway stem cell) by viral transduction or infection. Useful methods for viral transduction are described in, e.g., Wang et al., Gene Therapy, 2003, 10: 2105-2111.

In some embodiments, the polypeptide and/or nucleic acids of the gene modification system can be introduced into a target cell (e.g., an airway stem cell) using a delivery system. In certain instances, the delivery system comprises a nanoparticle, a microparticle (e.g., a polymer micropolymer), a liposome, a micelle, a virosome, a viral particle, a nucleic acid complex, a transfection agent, an electroporation agent (e.g., using a NEON transfection system), a nucleofection agent, a lipofection agent, and/or a buffer system that includes a nuclease component (as a polypeptide or encoded by an expression construct) and one or more nucleic acid components such as an sgRNA and/or a donor template. For instance, the components can be mixed with a lipofection agent such that they are encapsulated or packaged into cationic submicron oil-in-water emulsions. Alternatively, the components can be delivered without a delivery system, e.g., as an aqueous solution.

Methods of preparing liposomes and encapsulating polypeptides and nucleic acids in liposomes are described in, e.g., Methods and Protocols, Volume 1: Pharmaceutical Nanocarriers: Methods and Protocols. (ed. Weissig). Humana Press, 2009 and Heyes et al. (2005) J Controlled Release 107:276-87. Methods of preparing microparticles and encapsulating polypeptides and nucleic acids are described in, e.g., Functional Polymer Colloids and Microparticles volume 4 (Microspheres, microcapsules & liposomes). (eds. Arshady & Guyot). Citus Books, 2002 and Microparticulate Systems for the Delivery of Proteins and Vaccines. (eds. Cohen & Bernstein). CRC Press, 1996.

### EXAMPLES

### Example 1—Experimental Methods and Materials

EN Media: ADMEM/F12 supplemented with B27 supplement, nicotinamide (10 mM), human EGF (50 ng/mL), human Noggin (100 ng/mL), A83-01 (500 nM), N-acetylcysteine (1 mM), and HEPES (1 mM).

Cell Culture: Sinus tissue obtained from endoscopic surgery was cut into small pieces (1-2 mm²). Tissue pieces were washed with 10 mL sterile, PBS w/2× antibiotic/antimycotic on ice and digested with pronase (10 mg/mL, Sigma #P5147) for 2 h at 37° C. or at 4° C. overnight. Digestion was stopped using 1.0 mL FBS. Digested tissue was filtered through cell strainer (BD Falcon #352350) into a sterile 50 mL conical tube. The mixture was centrifuged at 950 rpm for 3 minutes at room temperature. RBC lysis was then performed using RBC lysis buffer (eBioscience™) as per manufacturer's instructions. After RBC lysis, cells were suspended in 1 mL EN media and counted. A small sample was fixed using 2% paraformaldehyde and permeabilized using Tris-buffered saline with 0.1% Tween 20. Cells were stained for cytokeratin 5 (Abcam, ab 193895). An isotype control (Abcam, ab 199093) was used to control for non-specific staining. Cytokeratin 5+ cells were plated at a density of 10,000 cells per cm² in tissue culture plates coated with 5% Matrigel. Cells were incubated at 37° C. in 5% 02 and 5% CO₂ in EN media with 10 μM ROCK inhibitor (Y-27632, Santa Cruz, sc-281642A). Cells obtained from CF patients were grown in EN media supplemented with additional antimicrobials for two days (Fluconazole—2 μg/mL, Amphotrecin B 1.25 μg/mL, Imipenem—12.5 μg/mL, Ciprofloxacin—40 μg/mL, Azithromycin—50 μg/mL, Meropenem—50 μg/mL). The concentration of antimicrobials was decreased 50% after 2-3 days and then withdrawn after editing (day 5-6).

Gene Editing: Media was replaced on day 3 and day 4 after plating from tissue. Gene correction was performed 5 days after plating. Cells were detached using Tryple. Cells were resuspended in OPTI-MEM at a density of 5 million cells/mL. Electroporation (Nucleofection) was performed using Lonza 4D 16-well Nucleocuvette™ Strips (Lonza, V4XP-3032). 6 μg of Cas9 (Integrated DNA Technologies, IA, Cat: 1074182) and 3.2 μg of MS-sgRNA (Trilink Biotechnologies, CA) (molar ratio=1:2.5) were complexed at room temperature for 10 minutes. 100,000 cells (20 μL of OPTI-MEM with 5 million cells/mL) were added to Cas9/MS-sgRNA mixture used per well and transferred to the strip. Cells were electroporated using the program CA-137. 80 μL of OPTI-MEM was added to each well after electroporation. Cells were transferred to a 12 well plate coated with 5% Matrigel (density=20,000 cells/cm²) and 400 μL of EN media with ROCK inhibitor was added. AAV carrying the correction template was added immediately after electroporation to maximize transduction. Multiplicity of Infection of 10⁶ particles per cell (as determined by qPCR) was optimal. AAV titers can also be determined by ddPCR which results in titers that are 10-fold lower. Media was replaced 48 h after electroporation. Gene correction levels were measured at least 4 days after electroporation.

Measuring Gene Correction: 4 days (or more) after gene correction, genomic DNA was extracted from cells using Quick Extract (Lucigen, QE09050) as per manufacturer's instructions. The ΔF508 locus was amplified using the primers: Forward: CCTTCTACTCAGTTTTAGTC (SEQ ID NO: 2) and Reverse: TGGGTAGTGTGAAGGGTTCAT (SEQ ID NO: 3). The PCR product was Sanger sequenced (primer: AGGCAAGTGAATCCTGAGCG (SEQ ID NO: 4)) and the percent of corrected alleles was determined using TIDER.

Air-Liquid Interface Culture of Corrected Sinus and Bronchial Basal cells: Gene corrected cells were plated 4-10 days after editing. 30,000 to 60,000 cells per well were plated in 6.5 mm Transwell plate with 0.4 μm pore polyester membrane insert. EN media was used to expand cells for 1-2 weeks. Once cells were confluent in Transwells and stopped translocating fluid, media in the bottom compartment was replaced with Pneumacult ALI media. For comparison, a small batch of cells were also cultured in media obtained from the University of North Carolina (UNC media)²⁰. The need for a coating of collagen IV was also tested.

Ussing Chamber Functional Assays: Ussing chamber measurements were performed 3-5 weeks after cells had stopped translocating fluid as described before. For Cl⁻ secretion experiments with sinus and human bronchial epithelial cells, solutions were as following in mM: Mucosal: NaGluconate 120, NaHCO₃ 25, KH₂PO₄ 3.3, K₂HPO₄ 0.8, Ca(Gluconate)² 4, Mg(Gluconate)² 1.2, Mannitol 10; Serosal: NaCl 120, NaHCO₃, 25, KH₂PO₄ 3.3, K₂HPO₄ 0.8, CaCl₂ 1.2, MgCl₂ 1.2, Glucose 10. The concentration of ion channel activators and inhibitors were as follows:
Amiloride—10 μM—Mucosal
Forskolin—10 μM—Bilateral
VX-770—10 μM—Mucosal
CFTRᵢₙₕ—172-20 μM—Mucosal
UTP—100 μM—Mucosal

Embedding Cells on pSIS Membrane: pSIS membranes (BiodesignR Sinonasal Repair Graft; COOK Medical, Bloomington, Ind.) were placed in 8 well confocal chambers. Sinus cells were seeded 4-8 days after electroporation. Four days after seeding, Calcein green (XuM) was added to cells. Cells were imaged using a dissection scope to identify densities that provided optimal coverage. SIS membranes with cells were fixed with 4% paraformaldehyde, permeabilized with TBS-T (0.1% Tween 20), and stained for cytokeratin 5 (ab193895). They were imaged using Leica SP8 confocal microscope.

### Example 2—Isolation and Culture of Sinus Basal Cells

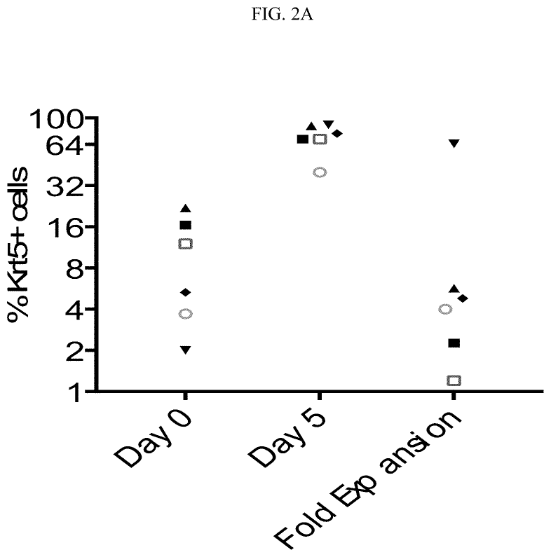

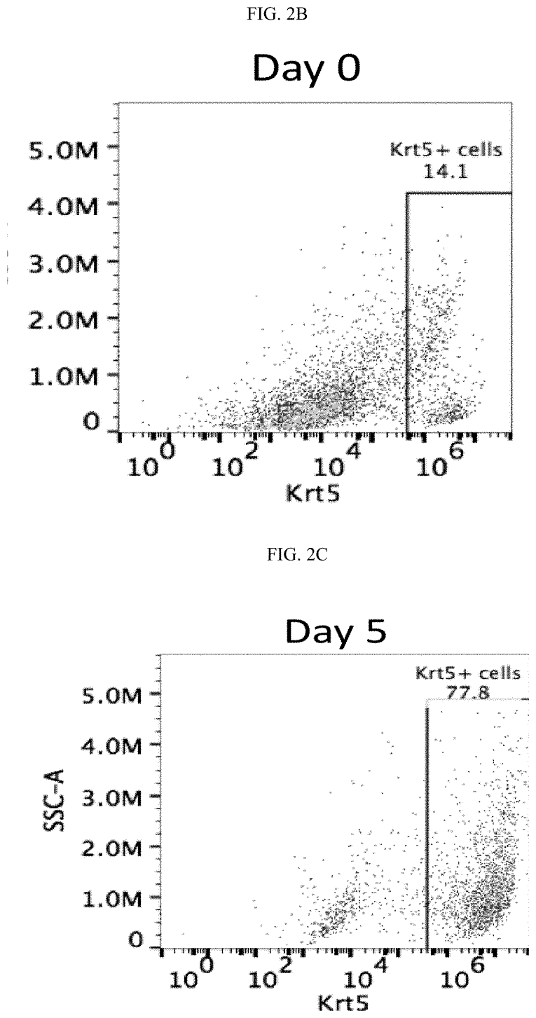

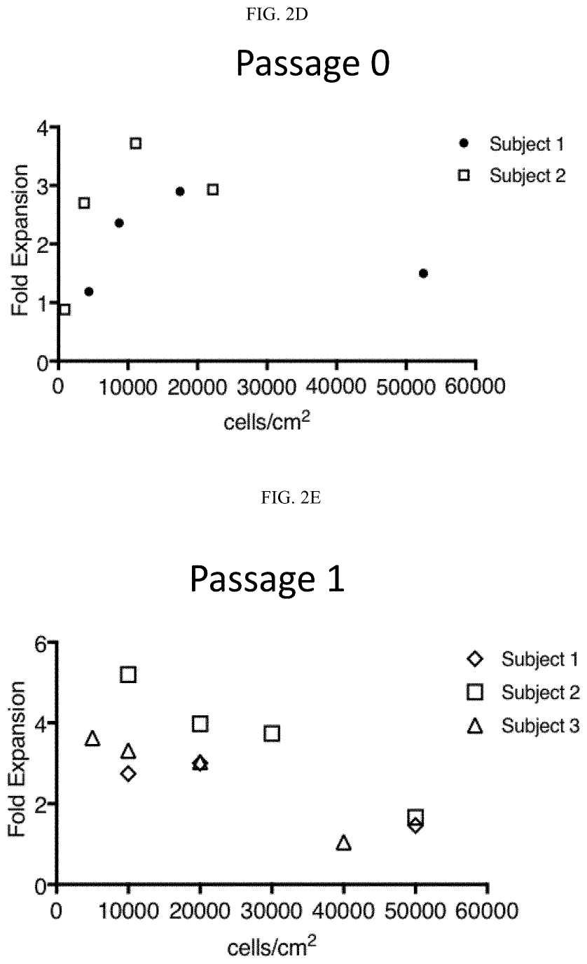

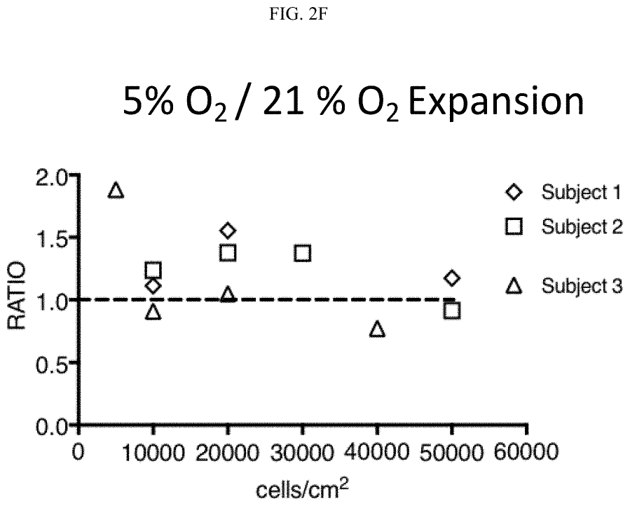

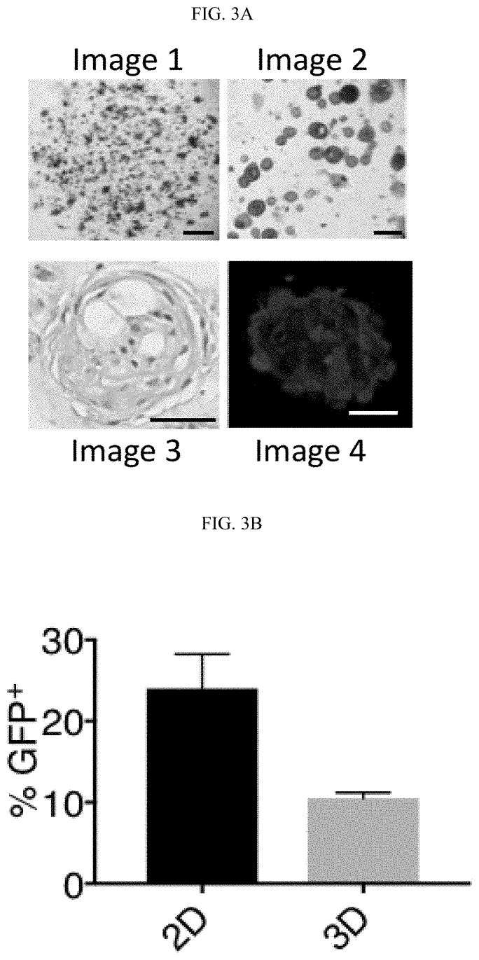

Sinus tissue was obtained from non-CF and CF patients undergoing functional endoscopic surgeries. After digestion with pronase, followed by red blood cell lysis, 2-22% cells were found to express cytokeratin 5 (Krt5⁻¹), a marker for stem/progenitor cells in sinus and lower airway epithelia (FIGS. 2A-2F).^{14,15} The cells were cultured in 5% Matrigel coated plates in the presence of Epidermal Growth Factor (EGF) along with BMP antagonist Noggin, the Transforming Growth Factor-β (TGF-β) inhibitor A83-01, and the Rho-kinase inhibitor Y-27632. FIGS. 2A-2F presents Krt5⁻¹ cells seen in 10 subjects on day 0 and enrichment of Krt5⁺ cells after 5 days in culture. Optimal cell density was about 10,000 cells/cm² both at P.0 and P.1. Culturing cells at 5% 02 also improved cell proliferation compared to 21% 02 (FIGS. 2A-2F). Cell editing was attempted on cells cultured as organoids and cells cultured as monolayers. A previously reported homologous recombination (HR) template (or donor template) expressing GFP at the CCRS locus (FIGS. 3A and 3B)¹⁰ was used. Cells cultured as monolayers showed higher HR. Hence, cells cultured as monolayers were used in subsequent experiments.

### Example 3—Insertion of Correction Sequence by Homologous Recombination in ΔF508 Locus

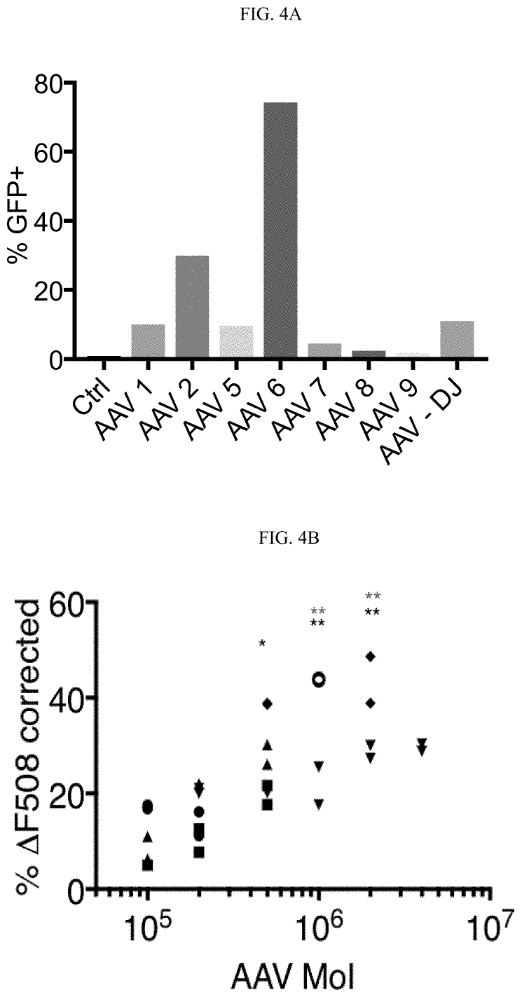

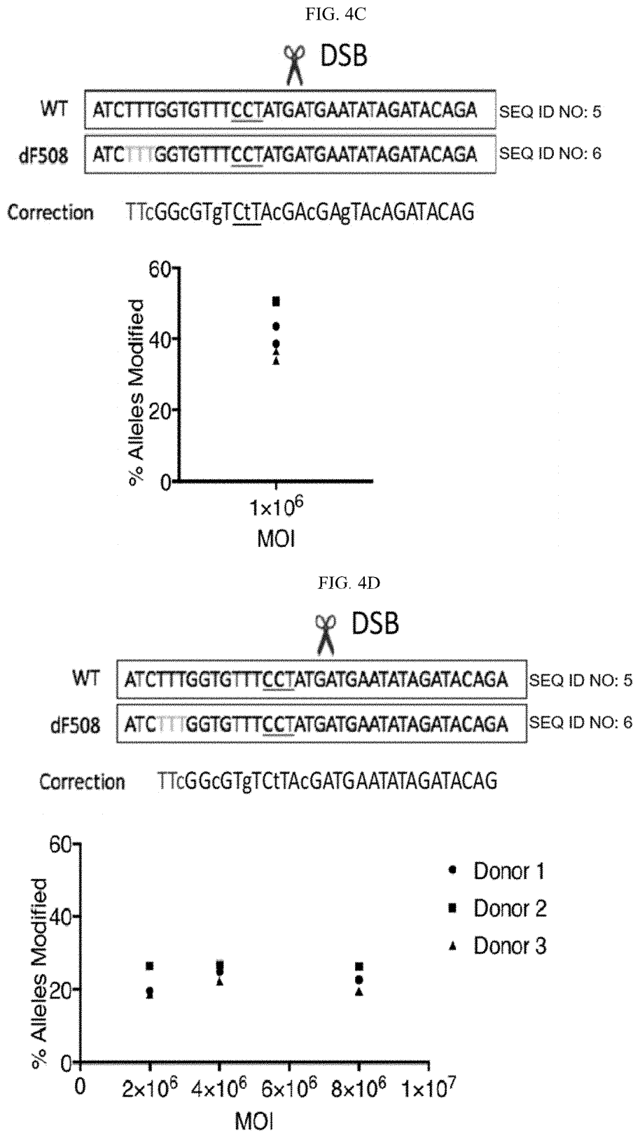

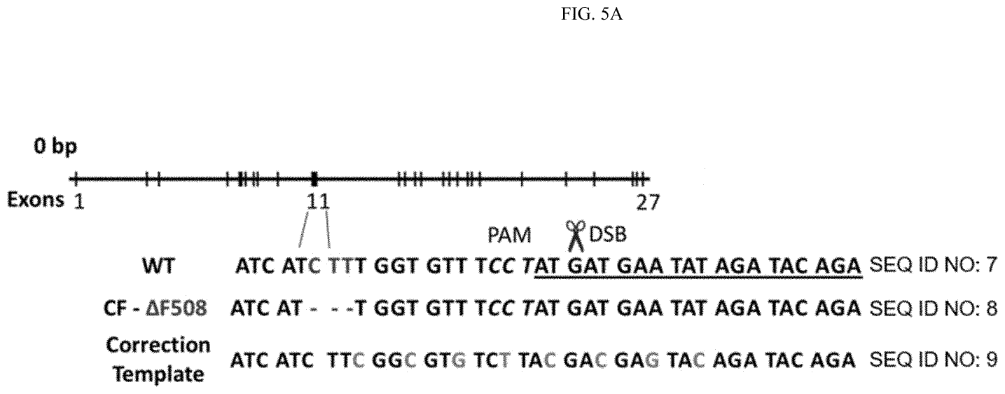

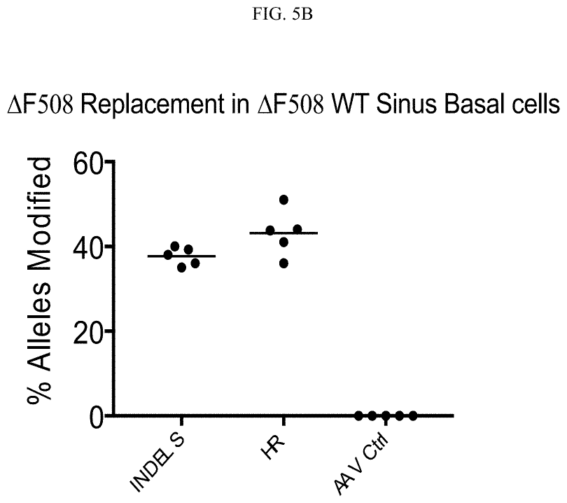

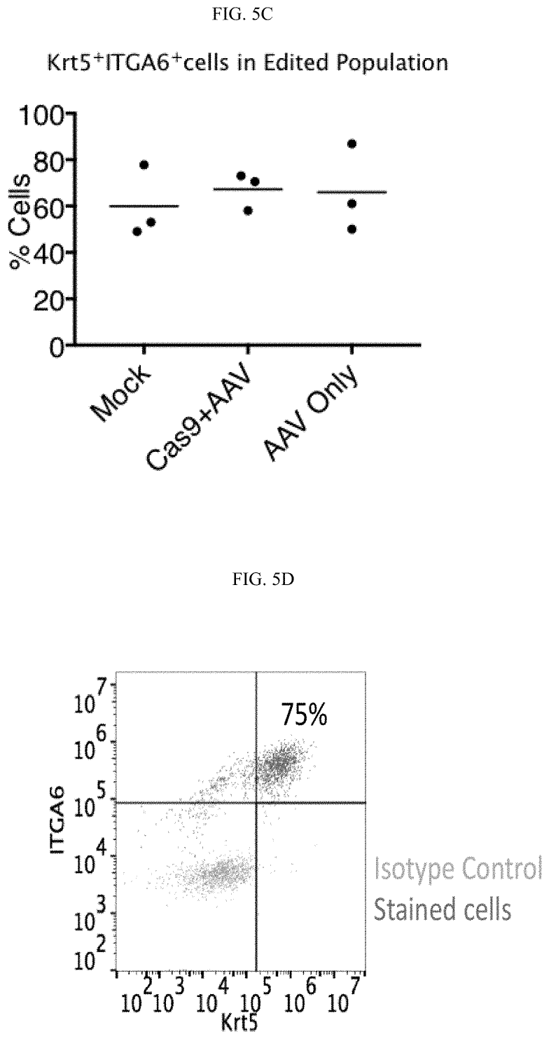

AAV6 at a multiplicity of infection (MOI) of 10⁶ particles per cell was found to have highest transduction among commonly used serotypes (FIGS. 4A and 4B). Five to six days after extraction from tissue, sinus basal cells were electroporated with Cas9 ribonuclear protein (RNP) and MS-sgRNA, followed by incubation with AAV6 containing a codon diverged sequence from CFTR exon 11 that includes the ΔF508 region (FIG. 5A). The CFTR exon 11 locus was amplified using junction PCR 4 days after editing. Insertions and deletions (INDELs) and recombination events (HR) were quantified using TIDER.¹⁶

The influence of the correction sequence on HR was tested. Correction sequence with 6 silent mutations surrounding the Cas9 double-stranded break was more effective than a correction sequence with 4 silent mutations (FIGS. 4C and 4D). While using the optimal template, the correction sequence was observed in 43±5% alleles (FIG. 5B) and INDELs were observed in 38±2% alleles (FIG. 5B). On the day of extraction 67±8% of edited cells were Krt5⁺ ITGA6+. This was similar to control (mock) cells, with 60±15% (FIGS. 5C and 5D). Thus, the corrected cells continued to display the phenotype associated with basal stem/progenitor cells after editing.^{15,17} The off-target activity of the MS-sgRNA is presented in Table 2. Off target activity of 0.17% was observed in OT-41 (Chr11:111971753-111971775). This region corresponds to an intron of the gene coding for the protein DIXDC1. DIXDC1 is a regulator of Wnt signaling and has been shown to be active in cardiac and neural tissue.¹⁸ The mismatch seen in OT-15 (both in control and edited cells at Chr10:17285197-17285219) is caused by a 28 bp insertion relative to the reference genome that occurs with a population wide allelic frequency of 8%. The region corresponds to a non-coding RNA.

TABLE 2
Off-target Activity of MS-SgRNA

| | Mock | Edited_Subject 1 | Edited_Subject 1 | Edited_Subject 2 |
|---|---|---|---|---|
| On | 0.00 | 73.63 | 80.87 | 77.12 |
| target | | | | |
| OT1 | 0.01 | 0.00 | 0.00 | 0.00 |
| OT2 | 0.00 | 0.00 | 0.00 | 0.00 |
| OT3 | 0.00 | 0.00 | 0.00 | 0.02 |
| OT4 | 0.00 | 0.00 | 0.00 | 0.00 |
| OT5 | 0.00 | 0.00 | 0.00 | 0.00 |
| OT6 | 0.00 | 0.00 | 0.01 | 0.01 |
| OT7 | 0.01 | 0.01 | 0.00 | 0.00 |
| OT8 | 0.00 | 0.00 | 0.00 | 0.00 |
| OT9 | 0.01 | 0.00 | 0.01 | 0.00 |
| OT10 | 0.01 | 0.00 | 0.01 | 0.01 |
| OT11 | 0.00 | 0.00 | 0.00 | 0.00 |
| OT12 | 0.00 | 0.00 | 0.01 | 0.00 |
| OT13 | 0.00 | 0.00 | 0.00 | 0.00 |
| OT14 | 0.00 | 0.00 | 0.00 | 0.00 |
| OT15 | 34.93 | 0.00 | 0.00 | 99.94 |
| OT16 | 0.00 | 0.01 | 0.09 | 0.00 |
| OT17 | 0.00 | 0.01 | 0.01 | 0.00 |
| OT18 | 0.00 | 0.01 | 0.01 | 0.00 |
| OT19 | 0.01 | 0.00 | 0.00 | 0.01 |
| OT20 | 0.01 | 0.01 | 0.01 | 0.00 |
| OT21 | 0.00 | 0.00 | 0.00 | 0.00 |
| OT22 | 0.01 | 0.01 | 0.01 | 0.02 |
| OT23 | 0.02 | 0.03 | 0.01 | 0.01 |
| OT24 | 0.01 | 0.00 | 0.00 | 0.00 |
| OT25 | 0.02 | 0.02 | 0.01 | 0.00 |
| OT26 | 0.01 | 0.00 | 0.00 | 0.03 |
| OT27 | 0.00 | 0.01 | 0.00 | 0.00 |
| OT28 | 0.00 | 0.00 | 0.01 | 0.00 |
| OT29 | 0.02 | 0.02 | 0.01 | 0.01 |
| OT30 | 0.00 | 0.02 | 0.02 | 0.01 |
| OT31 | 0.02 | 0.04 | 0.01 | 0.00 |
| OT32 | 0.01 | 0.02 | 0.01 | 0.02 |
| OT33 | 0.00 | 0.00 | 0.01 | 0.00 |
| OT34 | 0.00 | 0.00 | 0.00 | 0.00 |
| OT35 | 0.00 | 0.00 | 0.00 | 0.00 |
| OT36 | 0.02 | 0.01 | 0.00 | 0.01 |
| OT37 | 0.00 | 0.01 | 0.01 | 0.01 |
| OT38 | 0.00 | 0.01 | 0.01 | 0.00 |
| OT39 | 0.02 | 0.02 | 0.01 | 0.03 |
| OT40 | 0.02 | 0.01 | 0.01 | 0.00 |
| OT41 | 0.01 | 0.18 | 0.15 | 0.00 |
| OT42 | 0.00 | 0.00 | 0.00 | 0.00 |
| OT43 | 0.00 | 0.00 | 0.00 | 0.00 |
| OT44 | 0.00 | 0.00 | 0.01 | 0.01 |
| OT45 | 0.01 | 0.00 | 0.00 | 0.00 |
| OT46 | 0.03 | 0.01 | 0.00 | 0.07 |
| OT47 | 0.01 | 0.01 | 0.01 | 0.01 |

### Example 4—Correction of ΔF508 Mutation in Krt5⁺ Stem Cells Expanded from CF Patient Airway Epithelia

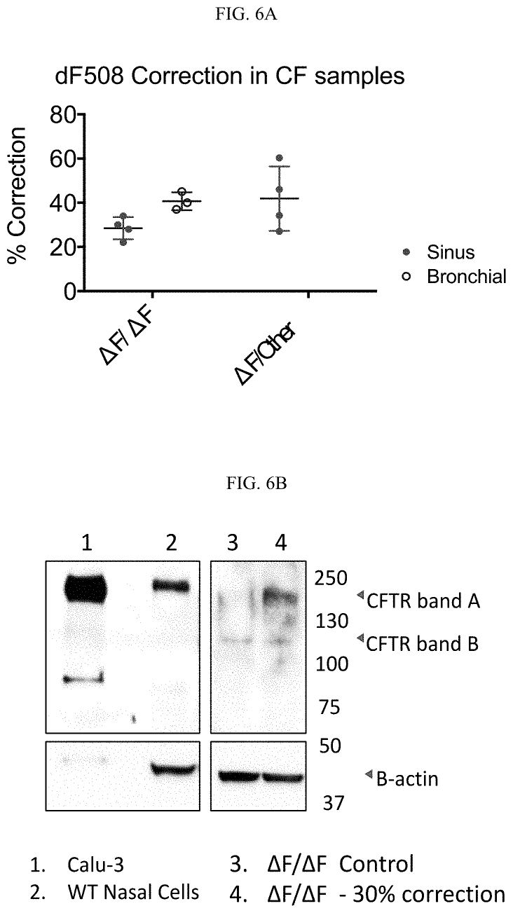

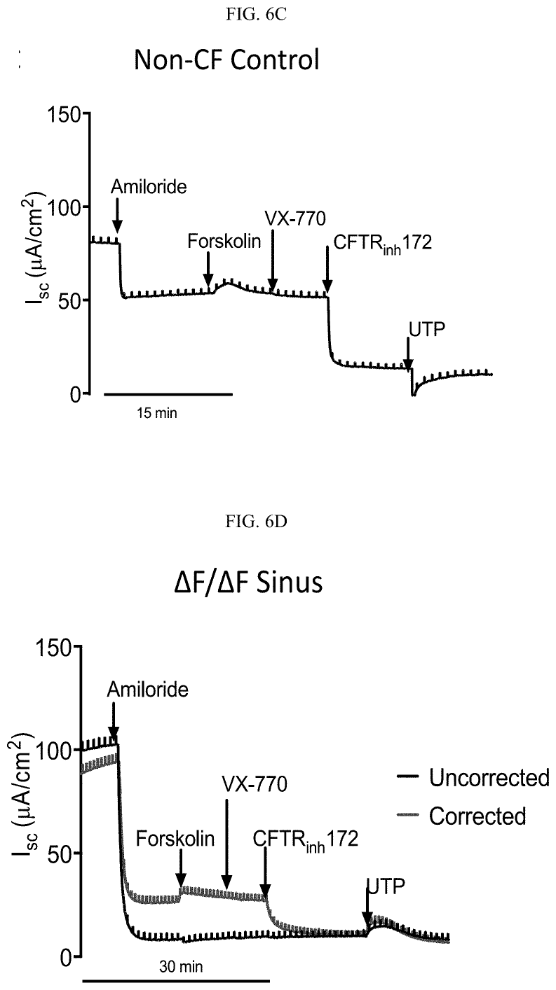

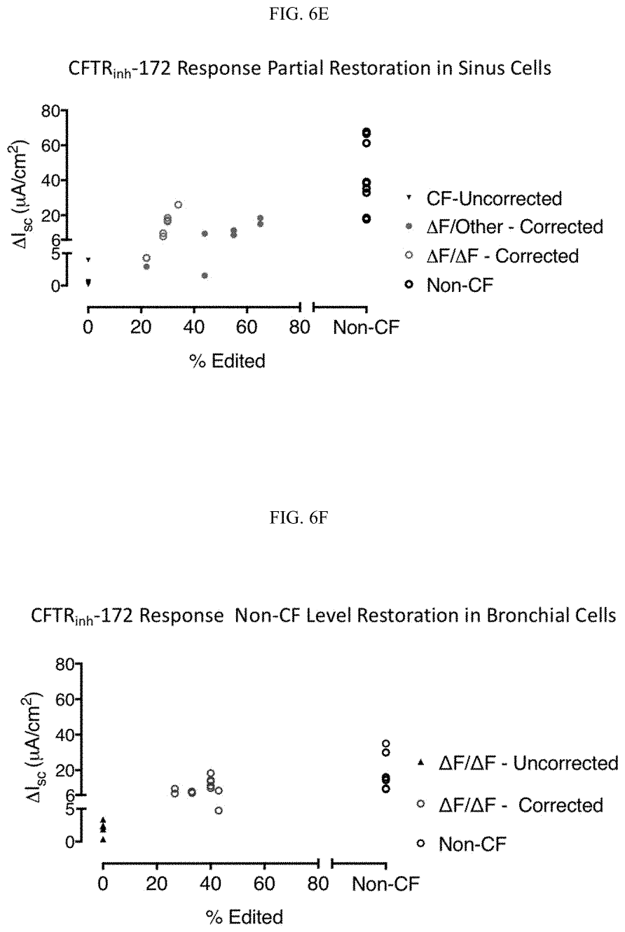

The optimized protocol was used to correct the ΔF508 mutation in sinus basal cells and bronchial basal cells (HBEC) from homozygous (ΔF/ΔF) patients and sinus basal cells from compound heterozygous (ΔF/other) patients. In sinus and bronchial basal cells from homozygous patients, allelic correction rates of 34±4% and 42±4% alleles, respectively, were observed. 41±15% allelic correction in compound heterozygous samples (FIG. 6A) was observed.

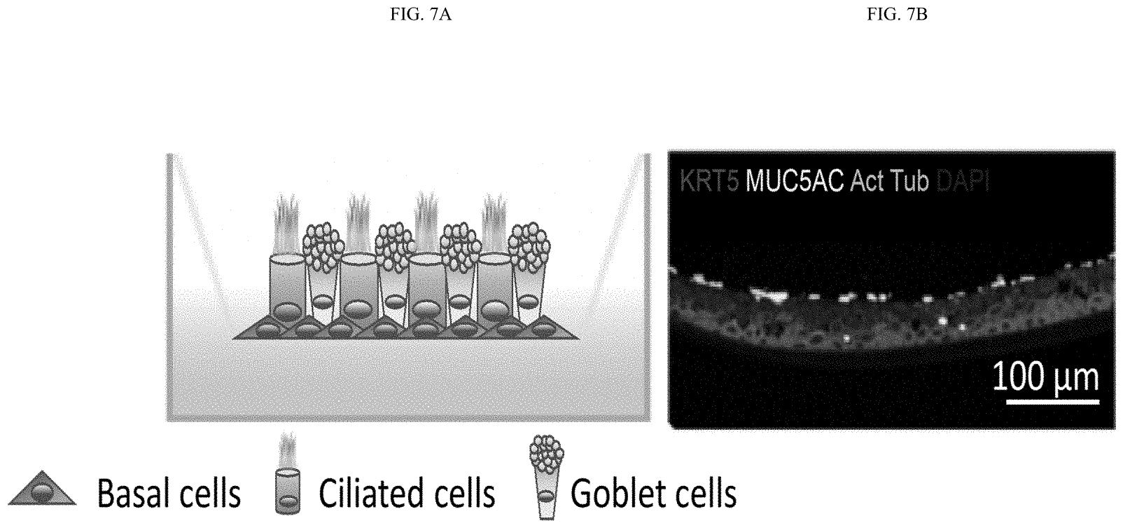

Corrected cells cultured at air-liquid interface using transwells resulted in a pseudostratified epithelium with a layer of Krt5⁺ basal cells, ciliated cells (acetylated alpha tubulin+), and mucus producing cells (MUC5AC+) (FIGS. 7A and 7B). The sinus basal cells were cultured at air-liquid interfaces (ALI) for 28-35 days. CFTR activity was measured using Ussing chamber assays. FIG. 6B shows a representative Western blot probing CFTR expression in non-CF, uncorrected and corrected CF sample after differentiation in ALI (CFTR Antibody 450). CFTR expression was not observed in the uncorrected homozygous sample (lane 3) and was restored in cells corrected using the Cas9/AAV platform (lane 4). CFTR expression in corrected cells was lesser than expression seen in non-CF nasal cells (lane 2).

Representative traces from non-CF and CF epithelial sheets are shown in FIGS. 6C and 6D. Consistent with the Western blot, corrected CF samples showed restored CFTR short-circuit current relative to uncorrected samples. CFTR short-circuit currents in corrected samples were lower than short-circuit currents in non-CF samples. The CFTRith-172-sensitive currents from corrected sinus and bronchial samples are plotted as a function of allelic correction in FIGS. 6E and 6F, respectively. The data were obtained from uncorrected and corrected CF samples belonging to homozygous (n=4 donors) and compound heterozygous patients (n=donors), as well as non-CF patients (n=3 donors).

Sinus cultures with higher editing efficiencies showed higher restoration of CFTR function. HBEC samples from 3 donors had similar correction rates and showed similar CFTRᵢₙₕ-172-sensitive currents. Non-CF sinus cultures showed average CFTRᵢₙₕ-172-sensitive short-circuit currents of 42±6 μA/cm² and CF sinus cultures with short-circuit currents of 0.8±0.04 μA/cm² (FIGS. 6C and 6D). Corrected CF sinus cultures showed CFTRᵢₙₕ-172 sensitive short-circuit currents of 12.2±2 μA/cm². Corrected HBECs showed average CFTRᵢₙₕ-172-sensitive currents of 10±1 μA/cm² compared to 2±1 μA/cm² seen in uncorrected ΔF508 homozygous HBECs and 18±3 μA/cm² seen in WT-HBECs (FIGS. 6C and 6D, FIGS. 1A and 1B). Overall, corrected sinus cultures showed CFTR currents that were 27±4% of non-CF cultures. Sheets derived from corrected HBECs showed CFTR currents 52±3% of non-CF HBECs. Genotype information, percent alleles corrected and change in CFTRᵢₙₕ-172 short-circuit currents for individual samples are presented in Table 3.

TABLE 3
Summary of Percent Allelic Correction of (ΔF508) in
CF Sinus Samples and Relative CFTR
Function with Respect to Non-CF and Uncorrected Controls

| Patient | Genotype | Editing | % Non-CF | Tissue |
|---|---|---|---|---|
| Patient 1 | ΔF/Other | 22 | 7 | Sinus |
| Patient 2 | ΔF/Other | 44 | 5 | Sinus |
| Patient 2 | ΔF/Other | 44 | 22 | Sinus |
| Patient 3 | ΔF/Other | 66 | 45 | Sinus |
| Patient 3 | ΔF/Other | 66 | 35 | Sinus |
| Patient 3 | ΔF/Other | 54 | 21 | Sinus |
| Patient 3 | ΔF/Other | 54 | 27 | Sinus |
| Patient 3 | ΔF/Other | 54 | 27 | Sinus |
| Patient 3 | ΔF/Other | 54 | 21 | Sinus |
| Patient 4 | ΔF/ΔF | 30 | 41 | Sinus |
| Patient 4 | ΔF/ΔF | 30 | 39 | Sinus |
| Patient 4 | ΔF/ΔF | 30 | 44 | Sinus |
| Patient 5 | ΔF/ΔF | 33 | 10 | Sinus |
| Patient 5 | ΔF/ΔF | 33 | 10 | Sinus |
| Patient 6 | ΔF/ΔF | 22 | 76 | Sinus |
| Patient 7 | ΔF/ΔF | 28 | 28 | Sinus |
| Patient 7 | ΔF/ΔF | 28 | 22 | Sinus |
| Patient 8 | ΔF/ΔF | 33 | 42 | Bronchial |
| Patient 8 | ΔF/ΔF | 26.7 | 37 | Bronchial |
| Patient 8 | ΔF/ΔF | 33.1 | 39 | Bronchial |
| Patient 8 | ΔF/ΔF | 43 | 30 | Bronchial |
| Patient 8 | ΔF/ΔF | 43 | 26 | Bronchial |
| Patient 8 | ΔF/ΔF | 43 | 46 | Bronchial |
| Patient 8 | ΔF/ΔF | 26.7 | 52 | Bronchial |
| Patient 8 | ΔF/ΔF | 33 | 43 | Bronchial |
| Patient 9 | ΔF/ΔF | 40 | 54 | Bronchial |
| Patient 9 | ΔF/ΔF | 40 | 99 | Bronchial |
| Patient 10 | ΔF/ΔF | 45 | 61 | Bronchial |
| Patient 10 | ΔF/ΔF | 45 | 78 | Bronchial |

### Example 5—Gene Edited Basal Cells can be Embedded in SIS Membrane

Lastly, genetically edited cells were embedded on a porcine SIS membrane that is already in clinical use for several indications, including sinonasal repair. The follow protocol was followed to culture cells prior to embedding: (1) Sinus tissue obtained from FESS was digested using Pronase, Collagenase, or Liberase. (2) RBC lysis was performed. Cells were counted and stained for cytokeratin 5 (Krt5). (3) Cells were plated at a density of 10,000 Krt5⁺ cells/cm² in tissue culture plates coated with 5% Matrigel and cultured at 5% 02 and 5% CO₂. (4) The base media used consisted of F12K media with epidermal growth factor, Noggin, TGF-beta inhibitor (A-83), and ROCK Inhibitor (Y-27632). (5) For the cystic fibrosis application: (a) cells were suspended in OPTI-MEM at a density of 5M cells/mL on day 5 and electroporated using Lonza 4D (Programs: CA137, CM119, DS120, CM150); (b) AAV was added within 15 minutes after electroporation which aided in the cellular uptake of the AAV; (c) dF508 mutations at the CFTR locus were corrected using sgRNA (UCUGUAUCUAUAUUCAUCAU (SEQ ID NO: 1)). The AAV correction template consisted of an 800 base pair (bp) left homology arm (LHA) upstream of the cut site, a 28 bp codon diverged correction template, followed by an 1800 bp right homology arm (RHA) as shown in SEQ ID NO: 10; (d) cells were plated at a density of 10,000 Krt5⁺ cells/cm² in tissue culture plates coated with 5% Matrigel and cultured at 5% 02 and 5% CO₂. (6) 4 days after editing, cells were embedded on SIS membrane at a density of 100,000 cells/cm².

SEQ ID NO: 10:
GCAAATTTCCTTTACACTCCACACTTATACCCCATTTCCTTTGTTTGTTT
ATTTGGTTTTTACTTCTAACTTTTCTTATTGTCAGGACATATAACATATT
TAAACTTTGTTTTTCAACTCGAATTCTGCCATTAGTTTTAATTTTTGTTC
ACAGTTATATAAATCTTTGTTCACTGATAGTCCTTTTGTACTATCATCTC
TTAAATGACTTTATACTCCAAGAAAGGCTCATGGGAACAATATTACCTGA
ATATGTCTCTATTACTTAATCTGTACCTAATAATATGAAGGTAATCTACT
TTGTAGGATTTCTGTGAAGATTAAATAAATTAATATAGTTAAAGCACATA
GAACAGCACTCGACACAGAGTGAGCACTTGGCAACTGTTAGCTGTTACTA
ACCTTTCCCATTCTTCCTCCAAACCTATTCCAACTATCTGAATCATGTGC
CCCTTCTCTGTGAACCTCTATCATAATACTTGTCACACTGTATTGTAATT
GTCTCTTTTACTTTCCCTTGTATCTTTTGTGCATAGCAGAGTACCTGAAA
CAGGAAGTATTTTAAATATTTTGAATCAAATGAGTTAATAGAATCTTTAC
AAATAAGAATATACACTTCTGCTTAGGATGATAATTGGAGGCAAGTGAAT
CCTGAGCGTGATTTGATAATGACCTAATAATGATGGGTTTTATTTCCAGA
CTTCACTTCTAATGGTGATTATGGGAGAACTGGAGCCTTCAGAGGGTAAA
ATTAAGCACAGTGGAAGAATTTCATTCTGTTCTCAGTTTTCCTGGATTAT
GCCTGGCACCATTAAAGAAAATATCATCTTcGGcGTgTCtTAcGAcGAgT
AcAGATACAGAAGCGTCATCAAAGCATGCCAACTAGAAGAGGTAAGAAAC
TATGTGAAAACTTTTTGATTATGCATATGAACCCTTCACACTACCCAAAT
TATATATTTGGCTCCATATTCAATCGGTTAGTCTACATATATTTATGTTT
CCTCTATGGGTAAGCTACTGTGAATGGATCAATTAATAAAACACATGACC
TATGCTTTAAGAAGCTTGCAAACACATGAAATAAATGCAATTTATTTTTT
AAATAATGGGTTCATTTGATCACAATAAATGCATTTTATGAAATGGTGAG
AATTTTGTTCACTCATTAGTGAGACAAACGTCCTCAATGGTTATTTATAT
GGCATGCATATAAGTGATATGTGGTATCTTTTTAAAAGATACCACAAAAT
ATGCATCTTTAAAAATATACTCCAAAAATTATTAAGATTATTTTAATAAT
TTTAATAATACTATAGCCTAATGGAATGAGCATTGATCTGCCAGCAGAGA
ATTAGAGGGGTAAAATTGTGAAGATATTGTATCCCTGGCTTTGAACAAAT
ACCATATAACTTCTAGTGACTGCAATTCTTTGATGCAGAGGCAAAATGAA
GATGATGTCATTACTCATTTCACAACAATATTGGAGAATGAGCTAATTAT
CTGAAAATTACATGAAGTATTCCAAGAGAAACCAGTATATGGATCTTGTG
CTGTTCACTATGTAAATTGTGTGATGGTGGGTTCAGTAGTTATTGCTGTA
AATGTTAGGGCAGGGAATATGTTACTATGAAGTTTATTGACAGTATACTC
CAAATAGTGTTTGTGATTCAAAAGCAATATCTTTGATAGTTGGCATTTGC
AATTCCTTTATATAATCTTTTATGAAAAAAATTGCAGAGAAAGTAAAATG
TAGCTTAAAATACAGTATCCAAAAAAATGGAAAAGGGCAAACCGTGGATT
AGATAGAAATGGCAATTCTTATAAAAAGGGTTGCATGCTTACATGAATGG
CTTTCCATGTATATACTCAGTCATTCAACAGTTTTTTTTTTAGAGCCCCA
TTCTTATTTTTTATACACTTTGAGAGCATAATGAAAAGAAAAGCTACCTG
CAAAAGTTTTGGACTTACCTCAAAGAGGATATACTTCATTCCTCAAAAGG
CCTTCTTCCAGGAATAGTATTTCATAACCTGGAGGTTGGAAAAATCTGGA
TTTGTTACAAAAAAATCTGAGTGTTTCTAGCGGACACAGATATTTGTCTA
GGAGGGGACTAGGTTGTAGCAGTGGTAGTGCCTTACAAGATAAATCATGG
GCTTTATTTACTTACGAGTGGAAAAGTTGCGGAAGGTGCCTTACAGACTT
TTTTTTTGCGTTAAGTATGTGTTTTCCCATAGGAATTAATTTATAAATGG
TGGTTTGATTTCCTCAAGTCAACCTTTAAAAGTATATTTAGCCAAAATAT
AGCTTAAATATATTACTAGTAATAAATTTAGTACTGTGGGTCTCTCATTC
TCAAAATGAGCATTTACTAATTTCTGAACACTGTGCTAGGTCCTGGGAAT
ACCAAATTGAATAAGACATAGTCTATTTTTCTGAAGGGTTTATAGCAGAG
TCCCCTGTGTTAATAATGAAGGAGTGTGTGGTATGTGAATCATATATCAA
TAGGGTTGTTAAAAATAATGAAAAAAGGAGAAGAGGAAGAACATCTTTTT
TTTTTCTGATTGCACGGGCAGCCTTAAAATTATTTTTGAAGTGTACAATT

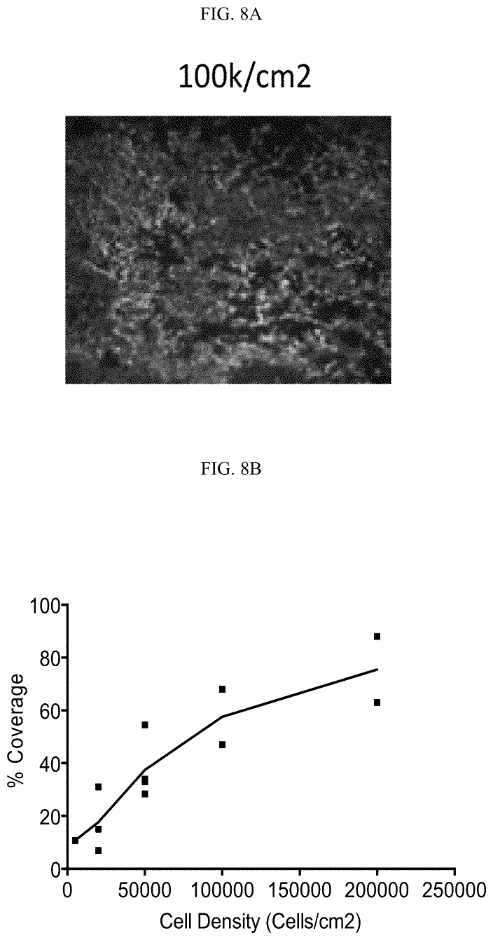

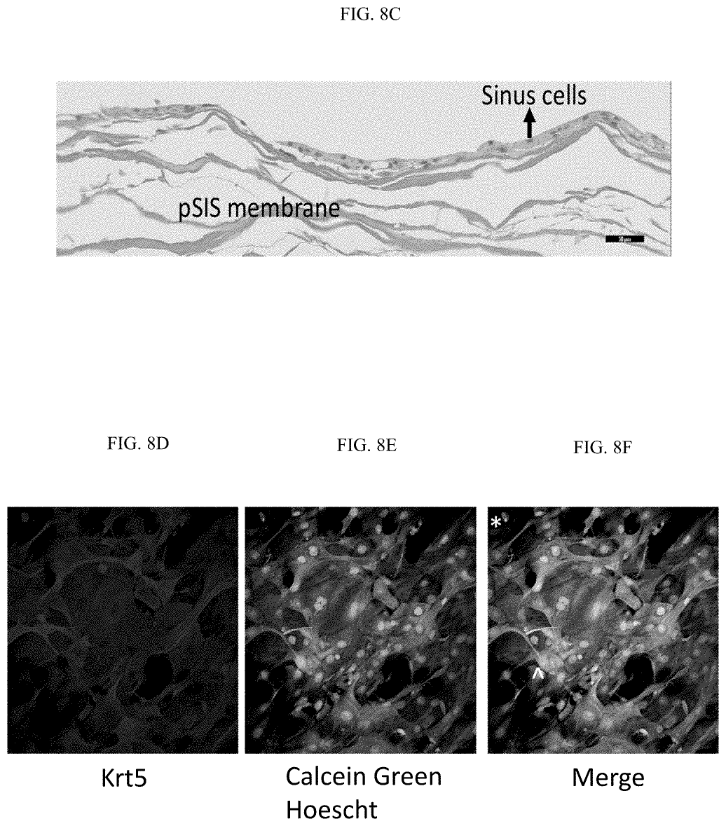

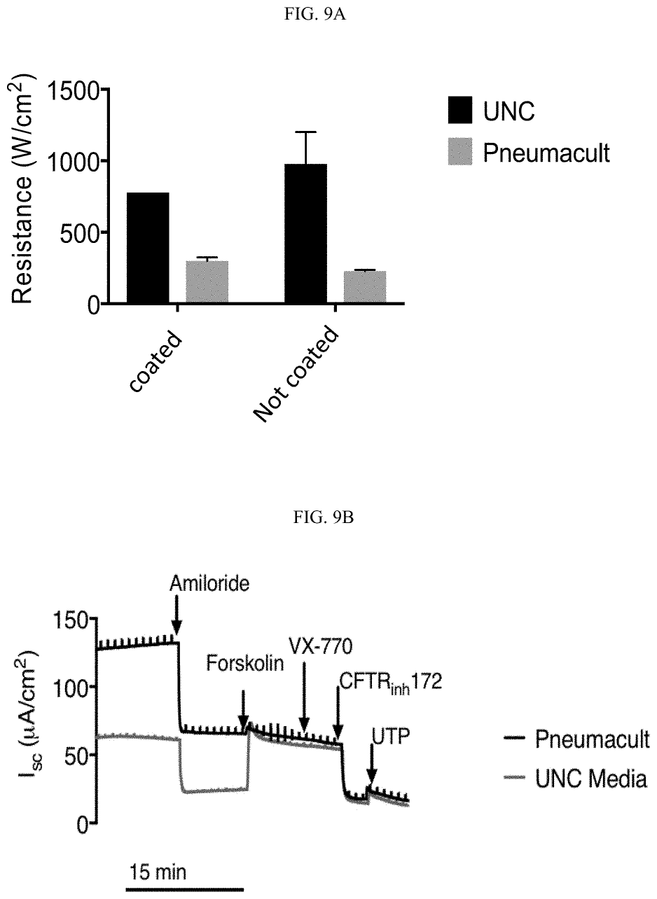

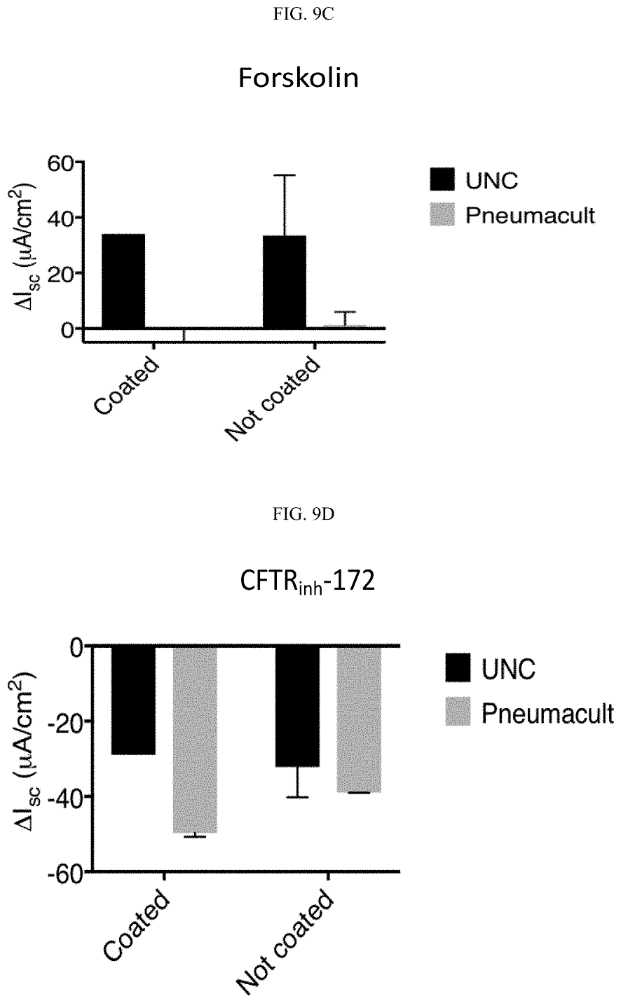

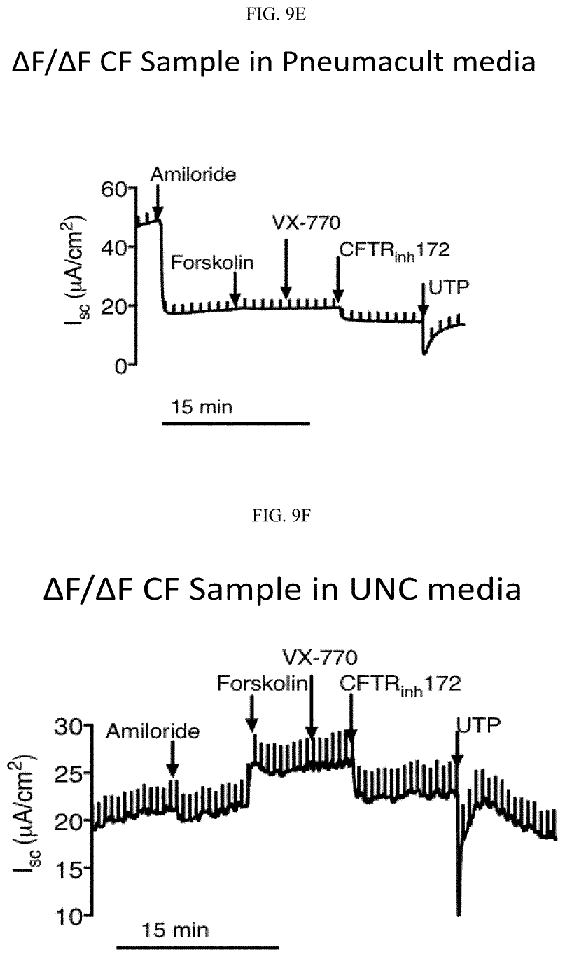

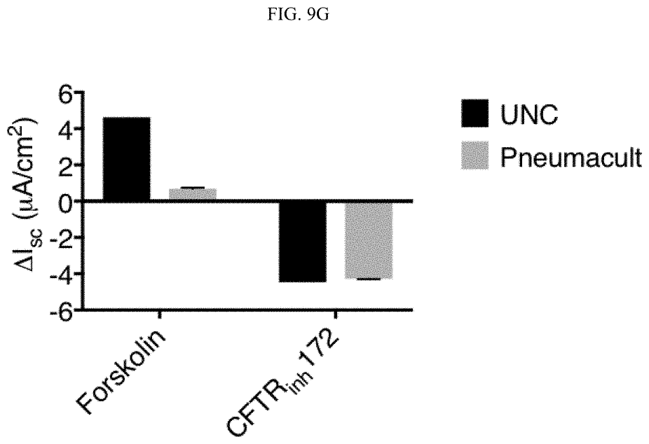

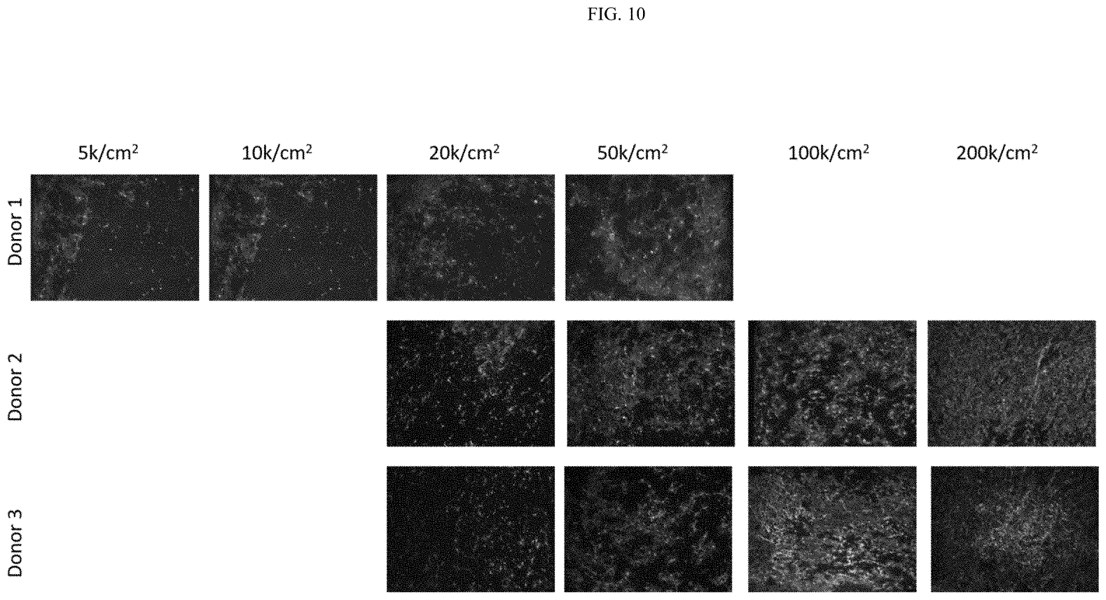

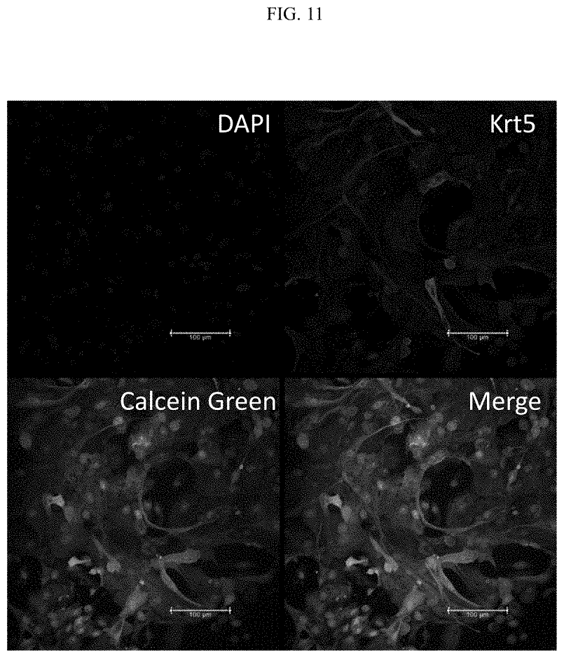

It was discovered that the optimal plating density to achieve 50-70% coverage in four days was greater than 50,000 cells/cm² (FIGS. 8A and 8B; FIG. 10). Cells seeded on pSIS membrane remained Krt5⁺ basal cells (FIG. 11). Mander's colocalization coefficients were calculated and the fraction of calcein green positive cells also positive for Krt5 (M1) was determined to be 53±15% (n=4 biological replicates) (FIGS. 8D-8F). The fraction of Krt5⁺ cells was dependent on the Krt5⁺ fraction measured on the day of seeding and did not change appreciably.

### Example 6—Cells Embedded in SIS Membrane can Differentiate

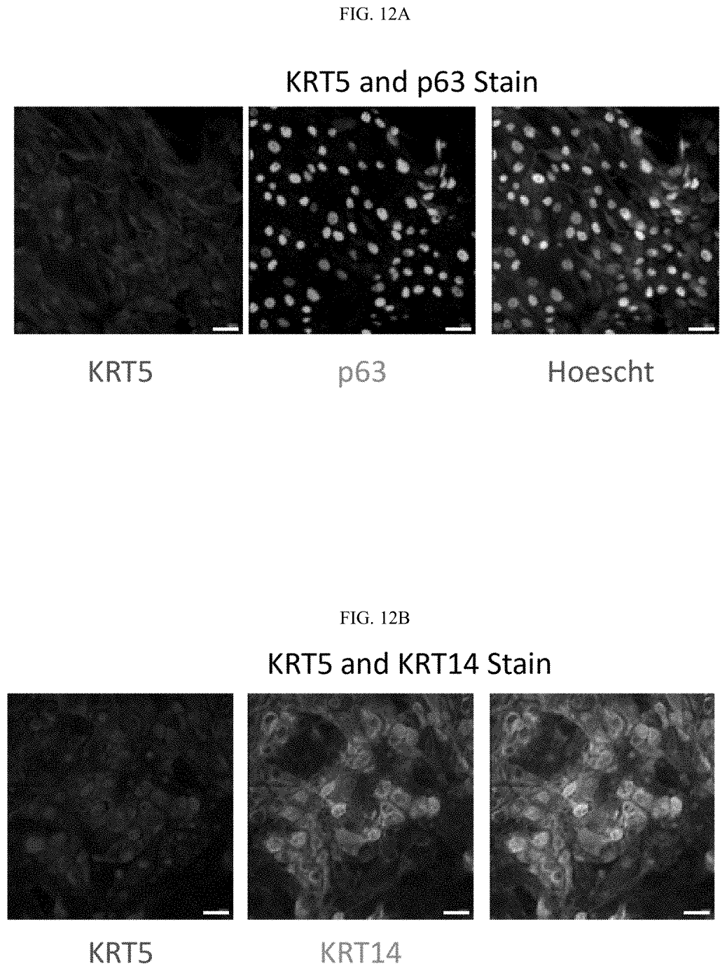

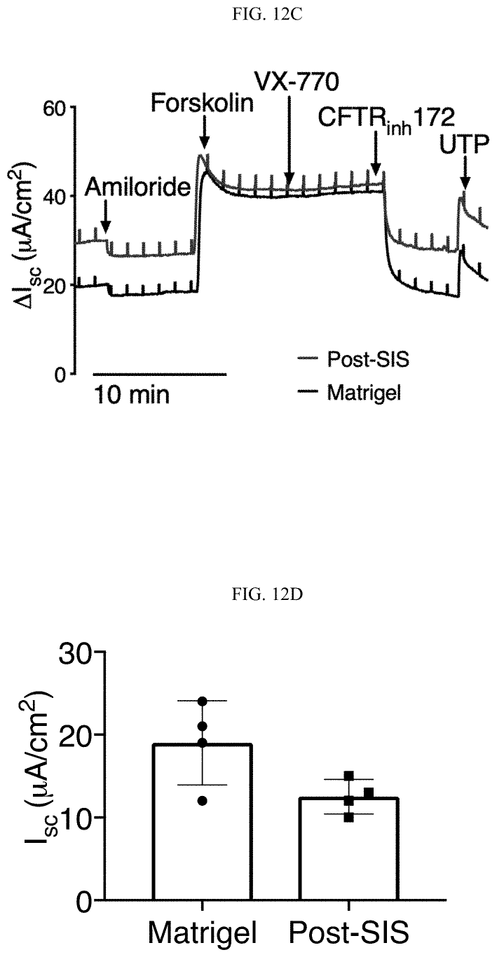

In this experiment, airway basal cells seeded on the SIS membrane were removed and differentiated on Transwells. Control basal cells cultured on Matrigel coated plates were also differentiated. The CFTR function in both differentiated cultures was measured (FIGS. 12C and 12D). The CFTR function was found to be not significantly different between the two groups. Thus, cells seeded on the SIS membrane retained their ability to differentiate and maintain CFTR function. FIGS. 12A and 12B further demonstrate that airway basal cells seeded on the SIS membrane also maintained stemness as shown by the markers p63 and cytokeratin 14.

### REFERENCES

1. Van Goor F, Hadida S, Grootenhuis P D J, Burton B, Stack J H, Straley K S, et al. Correction of the F508del-CFTR protein processing defect in vitro by the investigational drug VX-809. Proc Natl Acad Sci 2011; 108:18843-8.
2. Van Goor F, Hadida S, Grootenhuis P D J, Burton B, Cao D, Neuberger T, et al. Rescue of CF airway epithelial cell function in vitro by a CFTR potentiator, VX-770. Proc Natl Acad Sci 2009; 106:18825-30.
3. Wainwright C E, Elborn J S, Ramsey B W, Marigowda G, Huang X, Cipolli M, et al. Lumacaftor-Ivacaftor in Patients with Cystic Fibrosis Homozygous for Phe508del CFTR. N Engl J Med 2015; 373:220-31.
4. Schwank G, Koo B K, Sasselli V, Dekkers J F, Heo I, Demircan T, et al. Functional repair of CFTR by CRISPR/Cas9 in intestinal stem cell organoids of cystic fibrosis patients. Cell Stem Cell 2013; 13:653-8.
5. Crane A M, Kramer P, Bui J H, Chung W J, Li X S, Gonzalez-Garay M L, et al. Targeted correction and restored function of the CFTR gene in cystic fibrosis induced pluripotent stem cells. Stem Cell Reports 2015; 4:569-77.
6. Firth A L, Menon T, Parker G S, Qualls S J, Lewis B M, Ke E, et al. Functional Gene Correction for Cystic Fibrosis in Lung Epithelial Cells Generated from Patient iPSCs. Cell Rep 2015; 12:1385-90.
7. Armstrong D K, Cunningham S, Davies J C, Alton EWFW. Gene therapy in cystic fibrosis. Arch Dis Child 2014; 99:465 LP-468.
8. Charlesworth, C. T., Deshpande, P. S., Dever, D. P., Dejene, B., Gomez-Ospina, N., Mantri, S., Pavel-Dinu, M., Camarena, J., Weinberg, K. I. and Porteus M H. Identification of Pre-Existing Adaptive Immunity to Cas9 Proteins in Humans 2018.
9. Wang D, Mou H, Li S, Li Y, Hough S, Tran K, et al. Adenovirus-Mediated Somatic Genome Editing of Pten by CRISPR/Cas9 in Mouse Liver in Spite of Cas9-Specific Immune Responses. Hum Gene Ther 2015; 26:432-42.
10. Hendel A, Bak R O, Clark J T, Kennedy A B, Ryan D E, Roy S, et al. Chemically modified guide RNAs enhance CRISPR-Cas genome editing in human primary cells. Nat Biotechnol 2015; 33:985-9.
11. Dever D P, Bak R O, Reinisch A, Camarena J, Washington G, Nicolas C E, et al. CRISPR/Cas9 β-globin gene targeting in human haematopoietic stem cells. Nature 2016; 539:384-9.
12. Bak R O, Dever D P, Porteus M H. CRISPR/Cas9 genome editing in human hematopoietic stem cells. Nat Protoc 2018; 13:358-76.
13. Nayak J V., Rathor A, Grayson J W, Bravo D T, Velasquez N, Noel J, et al. Porcine small intestine submucosal grafts improve remucosalization and progenitor cell recruitment to sites of upper airway tissue remodeling. Int Forum Allergy Rhinol 2018; 0:1-7.
14. Bravo D T, Soudry E, Edward J A, Le W, Nguyen A L, Hwang P H, et al. Characterization of human upper airway epithelial progenitors. Int Forum Allergy Rhinol 2013; 3:841-7.
15. Wang D Y, Li Y, Yan Y, Li C, Shi L. Upper Airway Stem Cells: Understanding the Nose and Role for Future Cell Therapy. Curr Allergy Asthma Rep 2015; 15:1-9.
16. Brinkman E K, Kousholt A N, Harmsen T, Leemans C, Chen T, Jonkers J, et al. Easy quantification of template-directed CRISPR/Cas9 editing. Nucleic Acids Res 2018; 46:e58.
17. Bravo D T, Soudry E, Edward J A, Le W, Nguyen A L, Hwang P H, et al. Characterization of human upper airway epithelial progenitors. Int Forum Allergy Rhinol 2013; 3:841-7.
18. Wang X, Zheng L, Zeng Z, Zhou G, Chien J, Qian C, et al. DIXDC1 isoform, 1-DIXDC1, is a novel filamentous actin-binding protein. Biochem Biophys Res Commun 2006; 347:22-30.
19. Griesenbach U, Pytel K M, Alton E W F W. Cystic Fibrosis Gene Therapy in the U K and Elsewhere. Hum Gene Ther 2015; 26:266-75.
20. Gentzsch M, Boyles S E, Cheluvaraju C, Chaudhry I G, Quinney N L, Cho C, et al. Pharmacological rescue of conditionally reprogrammed cystic fibrosis bronchial epithelial cells. Am J Respir Cell Mol Biol 2017; 56:568-74.
21. Shah V S, Ernst S, Tang X X, Karp P H, Parker C P, Ostedgaard L S, et al. Relationships among CFTR expression, HCO 3-secretion, and host defense may inform gene- and cell-based cystic fibrosis therapies. Proc Natl Acad Sci 2016; 113:5382-7.
22. Johnson L G, Olsen J C, Sarkadi B, Moore K L, Swanstrom R, Boucher R C. Efficiency of gene transfer for restoration of normal airway epithelial function in cystic fibrosis. Nat Genet 1992; 2:21-5.
23. Char J E, Wolfe M H, Cho H J, Park I H, Jeong J H, Frisbee E, et al. A little CFTR goes a long way: CFTR-dependent sweat secretion from G551D and R117H-5T cystic fibrosis subjects taking ivacaftor. PLoS One 2014; 9:1-16.
24. De Nooijer R A, Nobel J M, Arets H G M, Bot A G, van Berkhout F T, de Rijke Y B, et al. Assessment of CFTR function in homozygous R117H-7T subjects. J Cyst Fibros 2011; 10:326-32.
25. McKone E F, Emerson S S, Edwards K L, Aitken M L. Effect of genotype on phenotype and mortality in cystic fibrosis: a retrospective cohort study. Lancet 2003; 361:1671-6.
26. Sheppard D N, Rich D P, Ostedgaard L S, Gregory R J, Smith A E, Welsh M J. Mutations in CFTR associated with mild-disease-form CI-channels with altered pore properties. Nature 1993; 362:160.
27. Rosen C, Shezen E, Aronovich A, Klionsky Y Z, Yaakov Y, Assayag M, et al. Preconditioning allows engraftment of mouse and human embryonic lung cells, enabling lung repair in mice. Nat Med 2015; 21:869.
28. Hansen S K, Rau M H, Johansen H K, Ciofu O, Jelsbak L, Yang L, et al. Evolution and diversification of Pseudomonas aeruginosa in the paranasal sinuses of cystic fibrosis children have implications for chronic lung infection. ISME J 2012; 6:31-45.
29. Figueiredo de Oliveira R A, Pedrazini M C, Wassail T. Relative area measurement of maxillary sinus by computed tomography 2014:111-6.

### Exemplary Embodiments

Exemplary embodiments provided in accordance with the presently disclosed subject matter include, but are not limited to, the claims and the following embodiments:
1. A composition for airway tissue regeneration, comprising an airway stem cell and a bioscaffold, wherein the airway stem cell expresses cytokeratin 5 (Krt5) and is embedded in the bioscaffold.
2. The composition of embodiment 1, wherein the bioscaffold comprises a decellularized extracellular matrix (ECM) membrane.
3. The composition of embodiment 1 or 2, wherein the airway stem cell expresses a wild-type Cystic Fibrosis Transmembrane Conductance Regulator (CFTR) protein.
4. The composition of any one of embodiments 1 to 3, wherein the airway stem cell is an upper airway stem cell.
5. The composition of embodiment 4, wherein the upper airway stem cell is an upper airway basal stem cell.
6. The composition of embodiment 5, wherein the upper airway basal stem cell is a sinus basal stem cell.
7. The composition of embodiment 1 or 3, wherein the airway stem cell is a bronchial stem cell.
8. The composition of embodiment 7, wherein the bronchial stem cell is a human bronchial epithelial cell (HBEC).
9. The composition of any one of embodiments 1 to 8, wherein the airway stem cell is a gene edited airway stem cell.
10. The composition of embodiment 9, wherein the gene edited airway stem cell is gene edited to correct an amino acid mutation in a protein.
11. The composition of embodiment 10, wherein the protein is a CFTR protein.
12. The composition of embodiment 10 or 11, wherein the gene edited airway stem cell is gene edited to correct an amino acid mutation at position 508 of a mutated CFTR protein.
13. The composition of any one of embodiments 9 to 12, wherein the gene edited airway stem cell is gene edited using a CRISPR (Clustered Regularly Interspaced Short Palindromic Repeats)/Cas (CRISPR-associated protein) nuclease system.
14. The composition of any one of embodiments 1 to 13, wherein the composition further comprises airway ciliated cells and/or airway mucus producing cells.
15. The composition of embodiment 14, wherein the airway ciliated cells express acetylated alpha tubulin.
16. The composition of embodiment 14 or 15, wherein the airway mucus producing cells express MUC5AC.
17. The composition of any one of embodiments 2 to 16, wherein the decellularized ECM membrane is derived from a tissue source selected from the group consisting of interstine tissue, pancreas tissue, liver tissue, lung tissue, trachea tissue, esophagus tissue, kidney tissue, bladder tissue, skin tissue, heart tissue, brain tissue, placenta tissue, and umbilical cord tissue.
18. The composition of any one of embodiments 2 to 17, wherein the decellularized ECM membrane is derived from a mammalian tissue source.
19. The composition of embodiment 18, wherein the decellularized ECM membrane is a porcine small intestinal submucosal (pSIS) membrane.
20. A method for airway tissue regeneration, comprising:
(a) inducing a stable gene modification of a target nucleic acid encoding a mutated protein in an airway stem cell via homologous recombination by introducing into the airway stem cell:
(1) a single guide RNA (sgRNA) comprising a first nucleotide sequence that is complementary to the target nucleic acid, and a second nucleotide sequence that interacts with a CRISPR-associated protein (Cas) polypeptide;
(2) a Cas polypeptide, an mRNA encoding a Cas polypeptide, and/or a recombinant expression vector comprising a nucleotide sequence encoding a Cas polypeptide, wherein the sgRNA guides the Cas polypeptide to the target nucleic acid; and
(3) a homologous donor adeno-associated viral (AAV) vector comprising a recombinant donor template comprising two nucleotide sequences comprising two non-overlapping, homologous portions of the target nucleic acid, wherein the nucleotide sequences are located at the 5′ and 3′ ends of a nucleotide sequence corresponding to the target nucleic acid to undergo homologous recombination;
(b) embedding the airway stem cell in a bioscaffold; and
(c) culturing the airway stem cell embedded in the bioscaffold, wherein the airway stem cell expresses Krt5.
21. The method of embodiment 20, wherein the bioscaffold comprises a decellularized ECM membrane.
22. The method of embodiment 20 or 21, wherein the mutated protein is a mutated CFTR protein and wherein the target nucleic acid is modified to encode a corresponding wild-type CFTR protein of the mutated CFTR protein in step (a).
23. The method of embodiment 22, wherein the mutated CFTR protein does not have a phenylalanine (F) at position 508.
24. The method of any one of embodiments 20 to 23, wherein the airway stem cell embedded in the bioscaffold differentiates into airway ciliated cells and/or airway mucus producing cells.
25. The method of embodiment 24, wherein the airway ciliated cells express acetylated alpha tubulin.
26. The method of embodiment 24, wherein the airway mucus producing cells express MUC5AC.
27. The method of any one of embodiments 20 to 26, wherein the homologous donor AAV vector is selected from a wild-type AAV serotype 1 (AAV1), wild-type AAV serotype 2 (AAV2), wild-type AAV serotype 3 (AAV3), wild-type AAV serotype 4 (AAV4), wild-type AAV serotype 5 (AAV5), wild-type AAV serotype 6 (AAV6), wild-type AAV serotype 7 (AAV7), wild-type AAV serotype 8 (AAV8), wild-type AAV serotype 9 (AAV9), wild-type AAV serotype 10 (AAV10), wild-type AAV serotype 11 (AAV11), wild-type AAV serotype 12 (AAV12), a variant thereof, and any shuffled chimera thereof
28. The method of embodiment 27, wherein the homologous donor AAV vector is a wild-type AAV6 or an AAV6 variant having at least 95% sequence identity to wild-type AAV6.
29. The method of any one of embodiments 20 to 28, wherein the airway stem cell comprises a population of airway stem cells.
30. The method of embodiment 29, wherein the stable gene modification of the target nucleic acid is induced in greater than about 70% of the population of airway stem cells.
31. The method of any one of embodiments 20 to 30, wherein the Cas polypeptide is a Cas9 polypeptide, a variant thereof, or a fragment thereof
32. The method of any one of embodiments 20 to 31, wherein the sgRNA comprises at least one modified nucleotide.
33. The method of any one of embodiments 22 to 32, wherein the sgRNA is used to correct a ΔF508 mutation in the mutated CFTR protein.
34. The method of embodiment 33, wherein the sgRNA comprises a sequence having at least 80% sequence identity to a sequence of UCUGUAUCUAUAUUCAUCAU (SEQ ID NO: 1).
35. The method of any one of embodiments 20 to 32, wherein the sgRNA and the Cas polypeptide are incubated together to form a ribonucleoprotein (RNP) complex prior to introducing into the airway stem cell.
36. The method of embodiment 35, wherein the RNP complex and the homologous donor AAV vector are concomitantly introduced into the airway stem cell.
37. The method of embodiment 35, wherein the RNP complex and the homologous donor AAV vector are sequentially introduced into the airway stem cell.
38. The method of embodiment 37, wherein the RNP complex is introduced into the airway stem cell before the homologous donor AAV vector.
39. The method of any one of embodiments 20 to 38, wherein the homologous donor AAV vector carries a sequence having at least 80% sequence identity to a sequence of SEQ ID NO: 10.
40. The method of any one of embodiments 20 to 38, wherein the sgRNA and the Cas polypeptide are introduced into the airway stem cell via electroporation.
41. The method of any one of embodiments 20 to 40, wherein the homologous donor AAV vector is introduced into the airway stem cell via transduction.
42. A method for treating an airway disease in a subject having a mutated protein, comprising grafting a composition comprising an airway stem cell and a bioscaffold, wherein the mutated protein causes the airway disease, the airway stem cell expresses Krt5 and a corresponding wild-type protein of the mutated protein, and the airway stem cell is embedded in the bioscaffold.
43. The method of embodiment 42, wherein the bioscaffold comprises a decellularized ECM membrane.
44. The method of embodiment 42 or 43, wherein the airway disease is cystic fibrosis (CF).
45. The method of any one of embodiments 42 to 44, wherein the mutated protein is a mutated CFTR protein.
46. The method of embodiment 45, wherein the mutated CFTR protein does not have a phenylalanine (F) at position 508.
47. The method of any one of embodiments 42 to 46, further comprising, prior to the grafting, isolating an airway stem cell from the subject having the mutated protein and gene editing the isolated airway stem cell to express a corresponding wild-type protein of the mutated protein.
48. The method of embodiment 47, comprising embedding the gene edited airway stem cell expressing the corresponding wild-type protein in the bioscaffold.
49. The method of embodiment 47 to 48, wherein the gene edited airway stem cell is edited using CRISPR (Clustered Regularly Interspaced Short Palindromic Repeats)/Cas (CRISPR-associated protein) nuclease system.
50. The method of any one of embodiments 42 to 46, further comprising, prior to the grafting, embedding the airway stem cell expressing Krt5 and the corresponding wild-type protein of the mutated protein in the bioscaffold.
51. The method of any one of embodiments 42 to 50, wherein the airway disease is selected from the group consisting of cystic fibrosis, chronic bronchitis, ciliary dyskinesia, bronchiectasis, chronic occlusive pulmonary disease (COPD), and diffuse panbronchiolitis.
52. The method of any one of embodiments 20 to 51, wherein the airway stem cell is an upper airway stem cell.
53. The method of embodiment 52, wherein the upper airway stem cell is an upper airway basal stem cell.
54. The method of embodiment 53, wherein the upper airway basal stem cell is a sinus basal stem cell.
55. The method of any one of embodiments 20 to 51, wherein the airway stem cell is a bronchial stem cell.
56. The method of embodiment 55, wherein the bronchial stem cell is a human bronchial epithelial cell (HBEC).
57. The method of any one of embodiments 21 to 41 and 43 to 56, wherein the decellularized ECM membrane is derived from a tissue source selected from the group consisting of interstine tissue, pancreas tissue, liver tissue, lung tissue, trachea tissue, esophagus tissue, kidney tissue, bladder tissue, skin tissue, heart tissue, brain tissue, placenta tissue, and umbilical cord tissue.
58. The method of any one of embodiments 21 to 41 and 43 to 57, wherein the decellularized ECM membrane is derived from a mammalian tissue source.
59. The method of embodiment 58, wherein the decellularized ECM membrane is a porcine small intestinal submucosal (pSIS) membrane.
60. An ex vivo regenerated airway stem cell produced by the method of any one of embodiments 20 to 41.

1. A composition for airway tissue regeneration, comprising an airway stem cell and a bioscaffold, wherein the airway stem cell expresses cytokeratin 5 (Krt5) and is embedded in the bioscaffold.

2. The composition of claim 1, wherein the bioscaffold comprises a decellularized extracellular matrix (ECM) membrane.

3. The composition of claim 1, wherein the airway stem cell expresses a wild-type Cystic Fibrosis Transmembrane Conductance Regulator (CFTR) protein.

4. The composition of claim 1, wherein the airway stem cell is an upper airway stem cell.

5-8. (canceled)

9. The composition of claim 1, wherein the airway stem cell is a gene edited airway stem cell.

10-11. (canceled)

12. The composition of claim 9, wherein the gene edited airway stem cell is gene edited to correct an amino acid mutation at position 508 of a mutated CFTR protein.

13. The composition of claim 9, wherein the gene edited airway stem cell is gene edited using a CRISPR (Clustered Regularly Interspaced Short Palindromic Repeats)/Cas (CRISPR-associated protein) nuclease system.

14. The composition of claim 1, wherein the composition further comprises airway ciliated cells and/or airway mucus producing cells.

15-16. (canceled)

17. The composition of claim 2, wherein the decellularized ECM membrane is derived from a tissue source selected from the group consisting of interstine tissue, pancreas tissue, liver tissue, lung tissue, trachea tissue, esophagus tissue, kidney tissue, bladder tissue, skin tissue, heart tissue, brain tissue, placenta tissue, and umbilical cord tissue.

18. The composition of claim 2, wherein the decellularized ECM membrane is derived from a mammalian tissue source.

19. The composition of claim 18, wherein the decellularized ECM membrane is a porcine small intestinal submucosal (pSIS) membrane.

20. A method for airway tissue regeneration, comprising:
(a) inducing a stable gene modification of a target nucleic acid encoding a mutated protein in an airway stem cell via homologous recombination by introducing into the airway stem cell:
(1) a single guide RNA (sgRNA) comprising a first nucleotide sequence that is complementary to the target nucleic acid, and a second nucleotide sequence that interacts with a CRISPR-associated protein (Cas) polypeptide;
(2) a Cas polypeptide, an mRNA encoding a Cas polypeptide, and/or a recombinant expression vector comprising a nucleotide sequence encoding a Cas polypeptide, wherein the sgRNA guides the Cas polypeptide to the target nucleic acid; and
(3) a homologous donor adeno-associated viral (AAV) vector comprising a recombinant donor template comprising two nucleotide sequences comprising two non-overlapping, homologous portions of the target nucleic acid, wherein the nucleotide sequences are located at the 5′ and 3′ ends of a nucleotide sequence corresponding to the target nucleic acid to undergo homologous recombination;
(b) embedding the airway stem cell in a bioscaffold; and
(c) culturing the airway stem cell embedded in the bioscaffold,
wherein the airway stem cell expresses Krt5.

21. The method of claim 20, wherein the bioscaffold comprises a decellularized ECM membrane.

22. The method of claim 20, wherein the mutated protein is a mutated CFTR protein and wherein the target nucleic acid is modified to encode a corresponding wild-type CFTR protein of the mutated CFTR protein in step (a).

23. The method of claim 22, wherein the mutated CFTR protein does not have a phenylalanine (F) at position 508.

24. The method of claim 20, wherein the airway stem cell embedded in the bioscaffold differentiates into airway ciliated cells and/or airway mucus producing cells.

25-26. (canceled)

27. The method of claim 20, wherein the homologous donor AAV vector is selected from a wild-type AAV serotype 1 (AAV1), wild-type AAV serotype 2 (AAV2), wild-type AAV serotype 3 (AAV3), wild-type AAV serotype 4 (AAV4), wild-type AAV serotype 5 (AAV5), wild-type AAV serotype 6 (AAV6), wild-type AAV serotype 7 (AAV7), wild-type AAV serotype 8 (AAV8), wild-type AAV serotype 9 (AAV9), wild-type AAV serotype 10 (AAV10), wild-type AAV serotype 11 (AAV11), wild-type AAV serotype 12 (AAV12), a variant thereof, and any shuffled chimera thereof.

28. The method of claim 27, wherein the homologous donor AAV vector is a wild-type AAV6 or an AAV6 variant having at least 95% sequence identity to wild-type AAV6.

29. The method of claim 20, wherein the airway stem cell comprises a population of airway stem cells.

30. The method of claim 29, wherein the stable gene modification of the target nucleic acid is induced in greater than about 70% of the population of airway stem cells.

31. The method of claim 20, wherein the Cas polypeptide is a Cas9 polypeptide, a variant thereof, or a fragment thereof.

32. The method of claim 20, wherein the sgRNA comprises at least one modified nucleotide.

33. The method of claim 22, wherein the sgRNA is used to correct a ΔF508 mutation in the mutated CFTR protein.

34. The method of claim 33, wherein the sgRNA comprises a sequence having at least 80% sequence identity to a sequence of UCUGUAUCUAUAUUCAUCAU (SEQ ID NO: 1).

35. The method of claim 20, wherein the sgRNA and the Cas polypeptide are incubated together to form a ribonucleoprotein (RNP) complex prior to introducing into the airway stem cell.

36-38. (canceled)

39. The method of claim 20, wherein the homologous donor AAV vector carries a sequence having at least 80% sequence identity to a sequence of SEQ ID NO: 10.

40-41. (canceled)

42. A method for treating an airway disease in a subject having a mutated protein, comprising grafting a composition comprising an airway stem cell and a bioscaffold, wherein the mutated protein causes the airway disease, the airway stem cell expresses Krt5 and a corresponding wild-type protein of the mutated protein, and the airway stem cell is embedded in the bioscaffold.

43-60. (canceled)


SEQUENCE LISTING (13 sequences)

SEQ ID NO: 1 - RNA, 20 nt, Artificial Sequence
  Description of Artificial Sequence Synthetic oligonucleotide
ucuguaucuauauucaucau

SEQ ID NO: 2 - DNA, 20 bp, Artificial Sequence
  Description of Artificial Sequence Synthetic primer
ccttctactcagttttagtc

SEQ ID NO: 3 - DNA, 21 bp, Artificial Sequence
  Description of Artificial Sequence Synthetic primer
tgggtagtgtgaagggttcat

SEQ ID NO: 4 - DNA, 20 bp, Artificial Sequence
  Description of Artificial Sequence Synthetic primer
aggcaagtgaatcctgagcg

SEQ ID NO: 5 - DNA, 36 bp, Homo sapiens
atctttggtgtttcctatgatgaatatagatacaga

SEQ ID NO: 6 - DNA, 33 bp, Artificial Sequence
  Description of Artificial Sequence Synthetic oligonucleotide
atcggtgtttcctatgatgaatatagatacaga

SEQ ID NO: 7 - DNA, 39 bp, Homo sapiens
atcatctttggtgtttcctatgatgaatatagatacaga

SEQ ID NO: 8 - DNA, 36 bp, Artificial Sequence
  Description of Artificial Sequence Synthetic oligonucleotide
atcattggtgtttcctatgatgaatatagatacaga

SEQ ID NO: 9 - DNA, 39 bp, Artificial Sequence
  Description of Artificial Sequence Synthetic oligonucleotide
atcatcttcggcgtgtcttacgacgagtacagatacaga

SEQ ID NO: 10 - DNA, 2600 bp, Artificial Sequence
  Description of Artificial Sequence Synthetic polynucleotide
gcaaatttcctttacactccacacttataccccatttcctttgtttgtttatttggtttttacttctaacttttcttattgtcaggacatataacatatttaaactttgtttttcaactcgaattctgccattagttttaatttttgttcacagttatataaatctttgttcactgatagtccttttgtactatcatctcttaaatgactttatactccaagaaaggctcatgggaacaatattacctgaatatgtctctattacttaatctgtacctaataatatgaaggtaatctactttgtaggatttctgtgaagattaaataaattaatatagttaaagcacatagaacagcactcgacacagagtgagcacttggcaactgttagctgttactaacctttcccattcttcctccaaacctattccaactatctgaatcatgtgccccttctctgtgaacctctatcataatacttgtcacactgtattgtaattgtctcttttactttcccttgtatcttttgtgcatagcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccagacttcacttctaatggtgattatgggagaactggagccttcagagggtaaaattaagcacagtggaagaatttcattctgttctcagttttcctggattatgcctggcaccattaaagaaaatatcatcttcggcgtgtcttacgacgagtacagatacagaagcgtcatcaaagcatgccaactagaagaggtaagaaactatgtgaaaactttttgattatgcatatgaacccttcacactacccaaattatatatttggctccatattcaatcggttagtctacatatatttatgtttcctctatgggtaagctactgtgaatggatcaattaataaaacacatgacctatgctttaagaagcttgcaaacacatgaaataaatgcaatttattttttaaataatgggttcatttgatcacaataaatgcattttatgaaatggtgagaattttgttcactcattagtgagacaaacgtcctcaatggttatttatatggcatgcatataagtgatatgtggtatctttttaaaagataccacaaaatatgcatctttaaaaatatactccaaaaattattaagattattttaataattttaataatactatagcctaatggaatgagcattgatctgccagcagagaattagaggggtaaaattgtgaagatattgtatccctggctttgaacaaataccatataacttctagtgactgcaattctttgatgcagaggcaaaatgaagatgatgtcattactcatttcacaacaatattggagaatgagctaattatctgaaaattacatgaagtattccaagagaaaccagtatatggatcttgtgctgttcactatgtaaattgtgtgatggtgggttcagtagttattgctgtaaatgttagggcagggaatatgttactatgaagtttattgacagtatactccaaatagtgtttgtgattcaaaagcaatatctttgatagttggcatttgcaattcctttatataatcttttatgaaaaaaattgcagagaaagtaaaatgtagcttaaaatacagtatccaaaaaaatggaaaagggcaaaccgtggattagatagaaatggcaattcttataaaaagggttgcatgcttacatgaatggctttccatgtatatactcagtcattcaacagttttttttttagagccccattcttattttttatacactttgagagcataatgaaaagaaaagctacctgcaaaagttttggacttacctcaaagaggatatacttcattcctcaaaaggccttcttccaggaatagtatttcataacctggaggttggaaaaatctggatttgttacaaaaaaatctgagtgtttctagcggacacagatatttgtctaggaggggactaggttgtagcagtggtagtgccttacaagataaatcatgggctttatttacttacgagtggaaaagttgcggaaggtgccttacagactttttttttgcgttaagtatgtgttttcccataggaattaatttataaatggtggtttgatttcctcaagtcaacctttaaaagtatatttagccaaaatatagcttaaatatattactagtaataaatttagtactgtgggtctctcattctcaaaatgagcatttactaatttctgaacactgtgctaggtcctgggaataccaaattgaataagacatagtctatttttctgaagggtttatagcagagtcccctgtgttaataatgaaggagtgtgtggtatgtgaatcatatatcaatagggttgttaaaaataatgaaaaaaggagaagaggaagaacatcttttttttttctgattgcacgggcagccttaaaattatttttgaagtgtacaatt

SEQ ID NO: 11 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 12 - DNA, 32 bp, Artificial Sequence
  Description of Artificial Sequence Synthetic oligonucleotide
ttcggcgtgtcttacgacgagtacagatacag

SEQ ID NO: 13 - DNA, 32 bp, Artificial Sequence
  Description of Artificial Sequence Synthetic oligonucleotide
ttcggcgtgtcttacgatgaatatagatacag